(19) United States
(12) Patent Application Publication
(10) Pub. No.: US 2019/0083676 A1
(43) Pub. Date: Mar. 21, 2019

(54) COMPOSITE BIOADHESIVE SEALANT

(71) Applicant: TECHNOLOGY INNOVATION MOMENTUM FUND (ISRAEL) LIMITED PARTNERSHIP, Tel-Aviv (IL)

(72) Inventors: Meital ZILBERMAN, Tel-Aviv (IL); Oded PINKAS, Petach-Tikva (IL)

(21) Appl. No.: 15/746,812

(22) PCT Filed: Oct. 20, 2016
(86) PCT No.: PCT/IL2016/051141

§ 371 Date: Jan. 23, 2018

Related U.S. Application Data
(60) Provisional application No. 62/247,213, filed on Oct. 28, 2015.

(51) Int. Cl. A61L 24/00; A61L 24/04

(52) U.S. Cl. CPC A61L 24/0089; A61L 2400/06; A61L 24/0015; A61L 24/043

(57) ABSTRACT

A kit and a bioadhesive, comprising gelatin, alginate, montmorillonite and a coupling agent, which is characterized by rapid curing, optimal viscosity, high burst strength, flexibility, biocompatibility and biodegradability, is disclosed.

### FIELD AND BACKGROUND OF THE INVENTION

The present invention, in some embodiments thereof, relates to bioadhesive materials, and more particularly, but not exclusively, to bioadhesive sealants, formulations and kits for forming same and to uses thereof.

Bioadhesive matrices, or bioadhesives for short, increasingly replace sutures and staples in medicine and surgery. The reasons for this increase include the potential speed with which internal surgical procedures might be accomplished; the ability of a bonding substance to effect complete closure, thus preventing seepage of fluids; the possibility of forming a bond without excessive deformation of the treated tissue; obviate the need for suture removal; cause less pain to the patient; its use requires simpler equipment which presents no risk of injury to the practitioner from sharp instruments; it provides lesser scar; and lowers the probability for infections. Bioadhesives may also be used for sealing air and body fluid leaks, e.g., fill fistula tract and as reinforcement of colorectal anastomosis in gastroenterological procedures, seal air leakage from the lungs, repair aortic dissections and seal blood vessels in bypass and other procedures, which may occasionally be resistant to conventional suture or stapling techniques; be used for topical wound closure; repair aortic dissections; and for internal and/or external fixation of certain devices.

Like any adhesive, bioadhesive matrices are formed upon curing a corresponding bioadhesive formulation. Thus, the formulation is applied onto e.g., a biological object, and when subjected to mixing, curing initiators or other curing initiating conditions, cures so as to afford the bioadhesive matrix.

Bioadhesives are required to be biocompatible and in most cases also biodegradable, and exhibit rapid curing, optimal bonding strength and elasticity once cured. Further, the bioadhesive matrices should be designed such that the corresponding formulation exhibits a workable consistency and curing/bonding time. More specifically, bioadhesive formulations should exhibit optimal initial viscosity, pliability and tack to allow adequate and easy application; not too fluid so as not to flow away from the site of use and not too viscous so as to interfere with even and proper application, and at the same time solidify quickly with short curing/gelation time, yet, not too short curing/gelation time, so as to allow smooth application to the desired site. In addition, bioadhesive formulations/matrices should exhibit an ability to bond rapidly to living tissue under wet conditions of bodily fluids; the bioadhesive matrix should form a bridge, typically a permeable flexible bridge; and the bioadhesive formulation, matrix and/or its metabolic (biodegradation) products should not cause local histotoxic or carcinogenic effects, while not interfering with the body's natural healing mechanisms.

One type of adhesive that is currently available is a cyanoacrylate adhesive. Cyanoacrylates, such as 2-octyl cyanoacrylate, known as Dermabond®, create a strong bond to tissue, enables rapid hemostasis and have the ability to polymerize in contact with fluids that are present at the biological surfaces. However, cyanoacrylate adhesives were found to be cytotoxic, the viscosity of the pre-cured adhesive formulation is too low and the cured cyanoacrylate matrix is stiff and non-biodegradable, interfering with normal wound healing. Hence, non-optimal viscosity, high flexural modulus and reports of cancer in animal experiments limited the use of cyanoacrylates to surface application on oral mucosa and life threatening arteriovenous.

Other known bioadhesive formulations are based on gelatin-resorcinol-formaldehyde, wherein a mixture of gelatin and resorcinol is warmed and crosslinked within tens of seconds by the addition of formaldehyde. The advantage of bioadhesives formed from such formulations is adequate bonding strength; however, cytotoxicity overshadows the advantages.

Another type of a currently available bioadhesive which is used as a tissue sealant utilizes components derived from bovine and/or human sources. For example, fibrin-based adhesive formulations are typically prepared by mixing a solution of fibrinogen and factor XIII with a solution of thrombin and CaCl₂. The two solutions are applied by a twin syringe equipped with a mixing nozzle, and the reaction is similar to white fibrin clot in blood clotting. Commercially available examples include Baxter Tisseel® and Ethicon Crosseal™. Advantages of fibrin-based bioadhesive matrices include hemostatic effect, biodegradability, good adherence to connective tissue and promotion of wound healing. Disadvantages include low strength (adhesive and cohesive), low viscosity (hard to apply only to the desired site) and risk of infection as in use of any human-origin product. In the United States fibrin adhesives are prepared from the patient's own blood in order to prevent contamination; however, this process is time consuming and expensive. Other limitations include air leakage in lung surgery that can reoccur a few days after surgery, possibly due to too-rapid absorption of the fibrin adhesive bridge.

Other known bioadhesives are protein-based tissue adhesives which are based on albumin or gelatin. The addition of polyamine, especially poly(lysine) or chitosan, or a polycarboxylate, especially citric acid or poly(acrylic acid), to increase the rate of crosslinking was also described. However, such bioadhesives are typically characterized by insufficient biocompatibility and strength.

Sung et al. [Journal of Biomedical Materials Research, Volume 46, Issue 4, pages 520-530, 15 Sep. 1999] report evaluation of various bioadhesive formulations including a formulation based on gelatin, alginate and carbodiimide. However, the formulations reported by Sung et al., are based on about 600 mg/ml gelatin content or higher, which do not afford a workable bioadhesive formulation.

U.S. Pat. No. 5,830,932 teaches an adhesive formulation suitable for making a barrier disc, an adhesive pad or wound treatment pad, comprising polyisobutylene, sodium alginate, pectin, gelatin, calcium silicate, and an absorptive agent such as cellulose.

WO 2013/121429, incorporated herein by reference as if full set forth herein, teaches a bioadhesive formulation comprising gelatin, alginate and a coupling agent, which can be used to replace sutures and staples as well as to release a bioactive agent sequestered therein.

The use of the clay mineral kaolinite (also known as kaolin) as a hemostatic agent incorporated into medical devices has been commercialized as QuikClot® [Kheirabadi, B. S. et al., J. Trauma., 2009, 67(3), pp. 450-459; Pahari, M. et al., Cath. Lab. Digest, 2010, 18(1), pp. 28-30; Trabattoni, D. et al., Int. J. Cardiol., 2012, 156(1), pp. 53-54; and Causey, M. W. et al., J. Surg Res., 2012, 177(2), pp. 301-305].

Gelatin and montmorillonite, a clay mineral, have been used in forming biodegradable nanocomposite films [Flaker, C. H. C et al., J. Food Eng., 2015, 167A, pp. 65-70; and Jorge, M. F. C. et al., Int. J. Polymer Sci., 2015, Article ID 806791].

Additional background art include Panzavolta S. et al. Journal of Applied Polymer Science, 2014, 131(11); Hsu, S. et al., Biorheology, 2007. 44(1): p. 17-28; Otani, Y. et al., Biomaterials, 1996. 17(14): p. 1387-1391; Bae, S. K. et al., Journal of Adhesion Science and Technology, 2002. 16(4): p. 361-372; Mo, X. et al., Journal of Biomaterials Science, Polymer Edition, 2000. 11(4): p. 341-351; McDermott, M. K., et al., Biomacromolecules, 2004. 5(4): p. 1270; Mo, X. et al., Journal of Biomedical Materials Research Part A, 2010. 94(1): p. 326-332; and Okino, H., et al., Journal of Biomedical Materials Research Part A, 2002. 59(2): p. 233-245.

### SUMMARY OF THE INVENTION

The present inventors have designed and successfully prepared and practiced bioadhesive formulations capable of forming bioadhesive matrices which are characterized by rapid curing, optimal viscosity, high bonding strength, flexibility, biocompatibility and biodegradability.

The bioadhesive formulations presented herein comprise gelatin, alginate, a coupling agent and a clay, and may further include a bioactive agent, for forming drug-eluting bioadhesive matrices.

The bioadhesive formulations and matrices presented herewith may be beneficially used in various biological and medical procedures.

Hence, according to an aspect of some embodiments of the present invention, there is provided a kit for forming a bioadhesive, which includes a first container containing a first formulation and a second container containing a second formulation, the first formulation includes gelatin and alginate and the second formulation includes a coupling agent for coupling the gelatin and/or for coupling the alginate and/or for coupling the gelatin to the alginate, wherein at least one of the first formulation and the second formulation which includes montmorillonite.

In some embodiments, the concentration of gelatin in a bioadhesive obtained by combining the first formulation and the second formulation at volume ratio of 1:9 to 25:1, ranges from 50 mg/ml to 500 mg/ml.

In some embodiments, the concentration of alginate in a bioadhesive obtained by combining the first formulation and the second formulation at volume ratio of 1:9 to 25:1, ranges from 5 mg/ml to 100 mg/ml.

In some embodiments, the concentration of montmorillonite (MMT) in a bioadhesive obtained by combining the first formulation and the second formulation at volume ratio of 1:9 to 25:1, ranges from 1 mg/ml to 50 mg/ml.

In some embodiments, the concentration of the coupling agent in a bioadhesive obtained by combining the first formulation and the second formulation at volume ratio of 1:9 to 25:1, ranges from 1 mg/ml to 40 mg/ml.

In some embodiments, in a bioadhesive obtained by combining the first formulation and the second formulation at volume ratio of 1:9 to 25:1, a concentration of the gelatin ranges from 200 mg/ml to 400 mg/ml, a concentration of the alginate ranges from 20 mg/ml to 40 mg/ml, a concentration of the montmorillonite ranges from 5 mg/ml to 30 mg/ml and a concentration of the coupling agent ranges from 10 mg/ml to 30 mg/ml.

In some embodiments, the first formulation and the second formulation include water.

In some embodiments, the quantity of water in a bioadhesive obtained by combining the first formulation and the second formulation at volume ratio of 1:9 to 25:1, ranges from 40% to 95% of the bioadhesive.

In some embodiments, the concentration of gelatin in a bioadhesive obtained by combining the first formulation and the second formulation at volume ratio of 1:9 to 25:1, is less than 500 mg/ml, and the bioadhesive is characterized by a room temperature viscosity that ranges from 1 Pa-sec to 50 Pa-sec upon combining and up to 30 minutes.

In some embodiments, the first formulation and/or the second formulation further include a crosslinking promoting agent.

In some embodiments, the first formulation and/or the second formulation further include a bioactive agent.

In some embodiments, the concentration of the bioactive agent in a bioadhesive obtained by combining the first formulation and the second formulation ranges from 0.1 percent weight per volume to 10 percent weight per volume of the total volume of the bioadhesive.

In some embodiments, the kit is for forming a bioadhesive matrix upon curing, wherein a curing time for forming the matrix ranges from 5 seconds to 30 minutes.

In some embodiments, the matrix is characterized by a burst strength expressed in a maximal pressure required to rupture a layer of the matrix having a thickness of about 1 mm and afforded by applying about 0.5 ml of the bioadhesive over and around a hole of about 3.0 mm uniform diameter punched in a collagen sheet, according to Standard Test Method for Burst Strength of Surgical Sealants ASTM F2392-04, the maximal pressure ranges from 350 mmHg to 650 mmHg.

In some embodiments, the kit is in the form of an applicator device for dispensing the first formulation from the first container and the second formulation from the second container to thereby form the bioadhesive.

In some embodiments, the applicator device includes:

the first container in a form of a first syringe having a first barrel defining a first chamber for retaining the first formulation, and a first plunger having one end received in the chamber for extruding the first formulation from the first chamber;

the second container in a form of a second syringe having a second barrel defining a second chamber for retaining the second formulation, and a second plunger having one end received in the second chamber extruding the second formulation from the second chamber;

a nozzle having a distal end, a proximal end, and a lumen extending through the nozzle and means for connecting the proximal end of the nozzle to the first chamber and the second chamber such that the first formulation and the second formulation come in contact in the lumen,

whereby forming the bioadhesive that may be ejected through the nozzle upon driving the first plunger and the second plunger.

In some embodiments, the kit presented herein is identified for use in adhering a biological object.

In some embodiments, the kit presented herein is identified for use in sealing a rupture in a biological object.

In some embodiments, the kit presented herein is identified for use in bonding at least two objects to one another, at least one of the objects being a biological object.

According to an aspect of some embodiments of the present invention, there is provided a use of the kit presented herein for forming a bioadhesive matrix.

In some embodiments, the bioadhesive matrix is for adhering a biological object.

In some embodiments, the bioadhesive matrix is for sealing a rupture in a biological object.

In some embodiments, the bioadhesive matrix is for bonding at least two objects to one another, at least one of the objects being a biological object.

According to an aspect of some embodiments of the present invention, there is provided a bioadhesive matrix, formed by contacting a first formulation that includes gelatin and alginate, and a second formulation that includes a coupling agent for coupling the gelatin and/or for coupling the alginate and/or for coupling the gelatin to the alginate, wherein at least one of the first formulation and the second formulation further include montmorillonite.

In some embodiments, the bioadhesive matrix further includes a bioactive agent sequestered therein, the bioadhesive matrix is a drug-eluting bioadhesive matrix.

According to an aspect of some embodiments of the present invention, there is provided a bioadhesive which includes:

a) gelatin;

b) alginate;

c) montmorillonite;

d) a coupling agent for coupling the gelatin and/or for coupling the alginate and/or for coupling the gelatin to the alginate; and

e) water.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying figures. With specific reference now to the figures in detail, it is stressed that the particulars shown are by way of example and for purposes of illustrative discussion of embodiments of the invention. In this regard, the description taken with the figures makes apparent to those skilled in the art how embodiments of the invention may be practiced.

In the figures:

FIGS. 1A-C present comparative bar plots showing the effect of the gelatin concentration on: the burst strength (FIG. 1A), the bonding strength in lap shear also showing comparison between manual application of the bioadhesive (dark bars in FIG. 1B) and application by a double-syringe (light bars in FIG. 1B) and the elastic modulus in compression (FIG. 1C), wherein the EDC concentration was kept constant at 20 mg/mL;

FIGS. 2A-C present comparative bar plots showing the effect of the alginate concentration on burst strength (FIG. 2A), the bonding strength in lap shear (FIG. 2B), and the elastic modulus in compression (FIG. 2C), of a bioadhesive based on 400 mg/mL gelatin and 20 mg/mL EDC;

FIGS. 3A-C present comparative bar plots showing the effect of the MMT concentration on the burst strength (FIG. 3A), the bonding strength in lap shear (FIG. 3B); and the elastic modulus in compression (FIG. 3C), of a bioadhesive based on 400:10:20 Gel-Al-EDC;

FIGS. 4A-C present comparative bar plots showing the effect of the kaolin concentration on the burst strength (FIG. 4A), the bonding strength in lap shear (FIG. 4B); and the elastic modulus in compression (FIG. 4C), of a bioadhesive based on 400:10:20 Gel-Al-EDC;

FIGS. 5A-B show the results of the XRD studies of MMT, wherein FIG. 5A shows the XRD patterns of pristine MMT (line No. 1) and unloaded bioadhesive (line No. 2), and FIG. 5B shows the normalized XRD patterns of a bioadhesive composite formulation, according to some embodiments of the present invention, loaded with 20 mg/ml MMT (line No. 3) 10 mg/ml MMT (line No. 4) and 5 mg/mL MMT (line No. 5);

FIGS. 6A-C present schematic illustrations of the chemical structure of kaolin (FIG. 6A) and sodium montmorillonite (MMT; FIG. 6B), and the different types of polymer/layered silicate composites, wherein a microcomposite is suggested to characterize the kaolin silicate composites, and an intercalated nanocomposite and exfoliated nanocomposite is suggested to characterize the MMT silicate composites;

FIGS. 7A-C present the results of rheological tests conducted to assess the viscosity of exemplary bioadhesive formulations, according to some embodiments of the present invention, showing the effect of the concentration of gelatin (FIG. 7A), alginate based on 400 mg/mL gelatin (FIG. 7B) and the concentration of MMT (marked by squares in FIG. 7C) and kaolin (marked by triangles in FIG. 7C), in a bioadhesive formulation having 400:10 Gel-Al;

FIG. 8 presents the gelation time of the 400:10:20 Gel-Al-EDC bioadhesive as affected by the concentration of MMT (marked by squares) and the concentration kaolin (marked by triangles), as used in an exemplary bioadhesive formulation based on 400:10:20 Gel-Al-EDC;

FIGS. 9A-B show the effect of coldwater fish gelatin (dark bars) versus porcine gelatin (light bars) on the initial viscosity (FIG. 9A) and the burst strength (FIG. 9B), measured for an exemplary bioadhesive formulation, according to some embodiments of the present invention, having Gel-Al-EDC 400-10-20 mg/mL; and

FIG. 10 presents a schematic illustration of a qualitative model describing the effects of the bioadhesive components on the cohesive and adhesive strength, wherein the dark/light arrows represent a case where an increase/decrease, respectively, in a certain parameter results in an increase in the following one, while the dashed lines represent a more moderate response.

### DESCRIPTION OF SPECIFIC EMBODIMENTS OF THE INVENTION

The present invention, in some embodiments thereof, relates to bioadhesive materials, and more particularly, but not exclusively, to bioadhesive sealants, formulations and kits for forming same and to uses thereof.

The principles and operation of the present invention may be better understood with reference to the figures and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As presented hereinabove, soft tissue bioadhesives are substances that hold tissues together, and could be broadly applicable in medicine and surgery, and can also be used for bleeding control and as sealants for used in broad medical applications. An ideal bioadhesive should allow rapid adhesion/sealing and maintain strong and close apposition of wound edges for an amount of time sufficient to allow wound healing. It should not interfere with body's natural healing mechanisms and should degrade without producing an excessive localized or generalized inflammatory response. In addition, it should be viscous liquid before curing (easy to apply) and solidify quickly with short gelation time.

While searching for improvements over the presently known bioadhesive formulations, the present inventors have surprisingly found that adding a clay mineral to a bioadhesive formulation based on gelatin, alginate and a coupling agent such as EDC, a highly effective bioadhesive sealant is afforded. It has been surprisingly found that while some of the most commonly used clay minerals, such as kaolin, which is also known for its coagulation-promotion activity, may be added to a bioadhesives, o montmorillonite (MMT) confers more advantageous properties to the bioadhesive.

As used herein, the term “bioadhesive” refers to a substance that can adhere to a living tissue and/or another object. A bioadhesive can therefore be used to seal a rupture in a living tissue, adhere two parts of a living (soft and/or hard) tissue, or adhere the living tissue to itself and/or to an inanimate object. According to some embodiments of the present invention, the term “bioadhesive” is used to describe a sticky and curable substance (glue) comprising naturally occurring polymers such as proteins and carbohydrates, which is capable of adhering also to living tissues and be used effectively and safely in medical applications, as discussed hereinabove. It is noted that in some applications the term “bioadhesive” is used to refer to formulations that are applied to external tissues or applied topically in order to affix an object to the skin, while in some applications where the bioadhesive is used internally, it is referred to as a “sealant”. Other than the properties shared by all bioadhesives, such as biocompatibility, pliability, curability, degradability and the like, when used as a sealant, the bioadhesive is required to exhibit mechanical properties that are relevant to the particular sealant uses, such as burst strength.

According to some embodiments, the uncured bioadhesive is referred to herein as a bioadhesive formulation while the cured bioadhesive is referred to herein as a bioadhesive matrix. As used herein, a bioadhesive formulation refers to a pliable formulation which can be applied to living tissues and/or objects to be adhered to the living tissue, with the aim of adhering the living tissues and/or the objects; and a bioadhesive matrix refers to the cured bioadhesive formulation that binds together the living tissues and/or the objects. The bioadhesive formulation, as referred to herein, is therefore a precursor of a corresponding bioadhesive matrix; the bioadhesive matrix is formed by curing the bioadhesive formulation, such that the bioadhesive formulation can be regarded as a pre-curing formulation.

Ingredients of the Bioadhesive:

A bioadhesives presented herein contain at least two types of polymers (e.g., alginate and gelatin) a coupling agent and MMT. The bioadhesive formulation undergoes a crosslinking reaction in the presence of the coupling agent, thereby curing.

Without being bound by any particular theory, it is assumed that the crosslinking reaction involves crosslinking of gelatin strands with alginate strands, essentially by coupling primary amines in the gelatin to carboxyl groups in the alginate, and/or with one or more other gelatin molecules. MMT is entrapped in the crosslinked polymer. When using a fluid formulation comprising gelatin as a major component and alginate as a minor component relative to gelatin, it is assumed that most of the crosslinks form between gelatin and alginate. This network of crosslinked polymers, being substantially gelatin and alginate entrapping MMT, referred to herein as a bioadhesive matrix, is the cured semi-solid, gel product of the fluid bioadhesive formulation. Hence, the matrix is regarded as a coupled gelatin-alginate matrix entrapping MMT afforded by coupling gelatin and alginate in the presence of MMT using a coupling agent.

As used herein, and is known in the art, the term “gelatin” describes a water-soluble protein that can form gel under certain conditions. Gelatin is typically obtained by heat dissolution at acidic or alkaline and partially hydrolyzing conditions of collagen. Type A gelatin is obtained by acidic process and has a high density of amino groups causing a positive charge. Type B gelatin is obtained by alkaline process and has high density of carboxyl groups causing negative charge. There are different sources for collagen such as animal skin and bone, which afford a variety of gelatin forms with a range of physical and chemical properties. Typically, gelatin contains eighteen amino acids that are linked in partially ordered fashion; glycine or alanine is about a third to half of the residues, proline or hydroxyproline are about one fourth and the remaining forth include acidic or basic amino acid residues. Typically, in order to dissolve gelatin in water it is necessary to reach a temperature of at least 35° C. by heating or stirring and adding hot water, depending on the source of gelatin used. Moderate heating enhances solubility and severe heating may cause aggregation or partial hydrolysis of gelatin. The viscosity of gelatin varies with type, concentration, time and temperature. Acid processed gelatin has slightly greater intrinsic viscosity compared to alkali processed gelatin. Gelatin is relatively cheap, it is biocompatible with negligible immunologic problems, and it is biodegradable. “Bloom” is a test to measure the strength of a gel or gelatin. The test determines the weight (in grams) needed by a probe (normally with a diameter of 0.5 inch) to deflect the surface of the gel 4 mm without breaking it. The result is expressed in Bloom grades or Bloom number, and it is typically between 30 and 300 Bloom. To perform the Bloom test on gelatin, a 6.67% gelatin solution is kept for 17-18 hours at 10° C. prior to being tested.

In the context of embodiments of the present invention, alternatives to gelatin may include non-animal gel sources such as agar-agar (a complex carbohydrate harvested from seaweed), carrageenan (a complex carbohydrate harvested from seaweed), pectin (a colloidal carbohydrate that occur in ripe fruit and vegetables), konjac (a colloidal carbohydrate extracted from plants of the genus Amorphophallus), guar gum (guaran, a type of galactomannan extracted from cluster beans of the genus Cyamopsis tetragonolobus) and various combinations thereof with or without gelatin.

As used herein, and is known in the art, the term “alginate” describes an anionic polysaccharide. Alginate, which is also referred to herein and in the art as alginic acid, is a block copolymer composed of β-D mannuronic acid monomers (M blocks) and α-L guluronic acid (G blocks), with different forms of alginate having different ratio of M/G. The term “alginate”, as used herein, encompasses various M/G ratio. M/G ratio varies according to the species, source and harvest season of the algae/plant.

In some embodiments, the alginate has an M/G ratio that ranges from 0.3 to 4, from 0.7 to 3, or from 1 to 2. In other embodiments, the M/G ratio is 0.7, 0.9, 1, 1.3, 1.5, 1.7, 1.9, 2, 2.3, 2.5, 2.7, 3, 3.5 or 4.

Alginate is known to form a viscous gum by binding water (capable of absorbing 200-300 times its own weight in water).

Alginate undergoes reversible gelation in aqueous solution under mild conditions through interactions with divalent cations that bind between G-blocks of adjacent alginate chains creating ionic inter chain bridges. Since alginate is generally anionic polymer with carboxyl end, it is known and used as a good mucoadhesive agent.

Naturally occurring alginate is typically produced in marine brown algae (e.g., Macrocystis pyrifera, Ascophyllum nodosum and Laminaria) and soil bacteria (Pseudomonas and Azotobacter). Synthetically prepared alginates are also contemplated.

Alginate is relatively cheap, it is biocompatible, evokes no immunologic response in mammals, and it is biodegradable.

In the context of some embodiments of the present invention, alginate can be used in a high-viscosity (HV) form, exhibiting more than 2 Pa-sec, or low-viscosity (LV) form, exhibiting 0.1-0.3 Pa-sec. As demonstrated in the Examples section hereinbelow, use of the LV/HV alginate forms adds another parameter to the fine-tuning and optimization of the bioadhesive formulation presented herein.

The bioadhesive further includes a clay mineral, which is typically added to the solution of gelatin/alginate mixture as a dry powder or a suspension of solid particles. Clay minerals include, without limitation, Kaolinite (Kaolin, Al₂Si₂O₅(OH)₄) Montmorillonite (MMT, (Na,Ca)_{0.33}(Al,Mg)₂Si₄O₁₀(OH)₂.nH₂O), Halloysite (Al₂Si₂O₅(OH)₄), Elite ((K,H₃O)(Al,Mg,Fe)₂(Si,Al)₄O₁₀[(OH)₂,(H₂O)]), Vermiculite ((MgFe,Al)₃(Al,Si)₄O₁₀(OH)₂.4H₂O), Talc (Mg₃Si₄O₁₀(OH)₂), Sepiolite (Mg₄Si₆O₁₅(OH)₂.6H₂O), Palygorskite (Attapulgite, (Mg,Al)₂Si₄O₁₀(OH).4(H₂O)) and Pyrophyllite (Al₂Si₄O₁₀(OH)₂).

Kaolinite (Kaolin) and Montmorillonite (MMT) are both layered silicates (phyllosilicates, or clay minerals) having a general form of parallel sheets of silicate tetrahedra of Si₂O₅ (aka 2:5 ratio) sandwiching a layer on another oxide such as alumina, and bonded by hydrogen bonds via water or hydroxyl groups. While kaolin is a 1:1 clay, meaning the silica and alumina layers alternate at a 1:1 ratio, MMT is a 2:1 clay, meaning that it has 2 tetrahedral silica sheets sandwiching a central octahedral oxide sheet. It has been observed that kaolin does not expand when wetted, while MMT has a remarkable wet expansion or swelling capacity. These structural differences may explain the difference in performance of the composite gelatin-alginate bioadhesive sealant formulations.

As discussed hereinabove, while kaolin is a well-known and widely used as a coagulant or coagulation-promoting agent in commercially available medical products, MMT is rarely used for such purposes. As demonstrated in the Examples section below, MMT was found advantageous in the context of embodiments of the present invention; for example, by conferring a higher burst strength to bioadhesives compared to kaolin. Hence, according to embodiments of the present invention, the bioadhesive comprises MMT.

The term “coupling agent”, as used herein, refers to a reagent that can catalyze or form a bond between two or more functional groups intra-molecularly, inter-molecularly or both. Coupling agents are widely used to increase polymeric networks and promote crosslinking between polymeric chains, hence, in the context of some embodiments of the present invention, the coupling agent is such that can promote crosslinking between polymeric chains; or such that can promote crosslinking between amino functional groups and carboxylic functional groups, or between other chemically compatible functional groups of polymeric chains; or is such that can promote crosslinking between gelatin and alginate. In some embodiments of the present invention the term “coupling agent” may be replaced with the term “crosslinking agent”. In some embodiments, one of the polymers serves as the coupling agent and acts as a crosslinking polymer.

By “chemically compatible” it is meant that two or more types of functional groups can react with one another so as to form a bond.

Exemplary functional groups which are typically present in gelatins and alginates include, but are not limited to, amines (mostly primary amines —NH₂), carboxyls (—CO₂H), sulfhydryls and hydroxyls (—SH and —OH respectively), and carbonyls (—COH aldehydes and —CO— ketones).

Primary amines occur at the N-terminus of polypeptide chains (called the alpha-amine), at the side chain of lysine (Lys, K) residues (the epsilon-amine), as found in gelatin, as well as in various naturally occurring polysaccharides and aminoglycosides. Because of its positive charge at physiologic conditions, primary amines are usually outward-facing (i.e., found on the outer surface) of proteins and other macromolecules; thus, they are usually accessible for conjugation.

Carboxyls occur at the C-terminus of polypeptide chain, at the side chains of aspartic acid (Asp, D) and glutamic acid (Glu, E), as well as in naturally occurring aminoglycosides and polysaccharides such as alginate. Like primary amines, carboxyls are usually on the surface of large polymeric compounds such as proteins and polysaccharides.

Sulfhydryls and hydroxyls occur in the side chain of cysteine (Cys, C) and serine, (Ser, S) respectively. Hydroxyls are abundant in polysaccharides and aminoglycosides.

Carbonyls as ketones or aldehydes can be form in glycoproteins, glycosides and polysaccharides by various oxidizing processes, synthetic and/or natural.

According to some embodiments of the present invention, the coupling agent can be selected according to the type of functional groups and the nature of the crosslinking bond that can be formed therebetween. For example, carboxyl coupling directly to an amine can be afforded using a carbodiimide type coupling agent, such as EDC; amines may be coupled to carboxyls, carbonyls and other reactive functional groups by N-hydroxysuccinimide esters (NHS-esters), imidoester, PFP-ester or hydroxymethyl phosphine; sulfhydryls may be coupled to carboxyls, carbonyls, amines and other reactive functional groups by maleimide, haloacetyl (bromo- or iodo-), pyridyldisulfide and vinyl sulfone; aldehydes as in oxidized carbohydrates, may be coupled to other reactive functional groups with hydrazide; and hydroxyl may be coupled to carboxyls, carbonyls, amines and other reactive functional groups with isocyanate.

Hence, suitable coupling agents that can be used in some embodiments of the present invention include, but are not limited to, carbodiimides, NHS-esters, imidoesters, PFP-esters or hydroxymethyl phosphines.

A carbodiimide is a complete crosslinker that facilitates the direct coupling (conjugation) of carboxyls to primary amines. Thus, unlike other reagents, carbodiimide is a zero-length crosslinker; it does not become part of the final crosslink between the coupled molecules. Because peptides, proteins, polysaccharides and aminoglycosides contain multiple carboxyls and amines, direct carbodiimide-mediated coupling/crosslinking usually causes random polymerization of polypeptides.

EDC, or N-(3-Dimethylaminopropyl)-N′-ethylcarbodiimide hydrochloride, is a widely used carbodiimide-type coupling agent and crosslinker which enables the condensation between carboxyl and amino groups to form amide bonds and the byproduct urea. Once reacted with amine/hydroxyl reactants, EDC is not present in the structure of the coupled product; hence its biocompatibility and biodegradability are not an issue in the context of the present embodiments. As a gelatin molecule exhibits both carboxyl and amino groups, this type of polymer may undergo intermolecular crosslinking by EDC.

It is known that EDC and its urea derivative are cytotoxic and inhibit cell growth. This high reactivity towards amino and carboxyl groups in the living tissues, as well as the release of a urea derivative, are probably the basis for EDC's cytotoxicity.

Alternatives for carbodiimide-type coupling agent, according to some embodiments of the present invention, include without limitation, glyoxal, formaldehyde, glutaraldehyde, polyglutaraldehyde, dextran, citric acid derivatives, microbial transglutaminase and genipin.

In some embodiments, the coupling agent is used up during the coupling reaction, and produces a urea derivative as a byproduct of the coupling reaction between amine and carboxyl groups. The nature of the urea derivative is determined by the nature of the coupling agent used.

According to some embodiments of the present invention, various coupling and crosslinking agents may be combined or used as additives in any given bioadhesive formulation based on gelatin and alginate and a coupling agent, so as to further promote the crosslinking reaction. In a representative example, NHS-esters are added to a carbodiimide-type coupling agent such as EDC.

The addition of NHS to the crosslinking reaction of EDC affords an NHS-activated carboxylic acid group, which is less susceptible to hydrolysis and prevents rearrangements. On the other hand, at high concentration NHS can react with the EDC and compete with the crosslinking reaction, thereby reducing the effective amount of EDC for crosslinking. Hence, reagents such as NHS are referred to herein a crosslinking promoting agents.

By adding various agents that promote the coupling reaction, and, in the context of the present invention, promote the formation of crosslinks in the forming bioadhesive matrix, it is intended to increase the crosslinking efficiency and/or reduce the amount of coupling agent needed to form a matrix the exhibits the desired characteristics, as discussed hereinabove. Hence, such agents are referred to herein as “crosslinking promoting agents”. The amount of a crosslinking promoting agent is given as weight/volume per weight/volume percent (w/v/w/v), i.e. relative to the amount of the coupling agent, and according to some embodiments of the present invention, this amount ranges from about 1% to 100%, or from 1% to 200% weight/volume per weight/volume percent.

Representative examples of crosslinking promoting agents include, without limitation, sulfo-NHS, HOBt, HOAt, HBtU, HCtU, HAtU, TBtU, PyBOP, DIC pentafluorophenol and the likes.

According to some embodiments of the present invention, a combination of a crosslinking agent and a crosslinking promoting agent in the bioadhesive formulations presented herein, affords bioadhesive matrices with improved bonding strength. Furthermore, the combination of a crosslinking agent such as EDC and a crosslinking promoting agent such as N-hydroxysuccinimide (NHS), allows for a significant reduction in the amount of EDC in the bioadhesive. A reduction of the amount of EDC is beneficial due to the medical safety and cytotoxicity implications of using EDC.

In some embodiments, the amount of the crosslinking promoting agent may be 1, 2, 3, 4, 5, 6, 7, 8, 9, 10, 12, 14, 16, 18, 20, 25, 30, 35, 40, 45, 50, 60, 70, 100, 150, 200%, including any value between 1 and 200% relative to the amount of the coupling agent, or can be even higher. In some embodiments of the present invention, the amount of the crosslinking promoting agent is 5, 10, 15, 20, 30 or 40% relative to the amount of the coupling agent, including any value from 5 to 40.

When used in tandem, EDC and NHS afford stronger bonding bioadhesive matrices, even when relatively low concentration of EDC is used. An exemplary formulation comprises an amount of EDC which is 10 mg/ml and an amount of NHS which is 10% relative to the amount of the EDC.

Various additional and optional additives may be added to the bioadhesive in order to modify its pre-curing characteristics, such as for example, viscosity modifiers for improved application and spread confinement, penetration enhancers, and colorants or fluorescent agents for allowing tracking during application and follow-up. Various additives may be added to the bioadhesive in order to modify its post-curing characteristics, namely additives that affect the characteristics of the resulting matrix, such as for example, additional coupling/crosslinking agents, calcium ions and ions of other earth-metals, which act as gelling agents by virtue of being crosslinkers for various alginate species, plasticizes, hardeners, softeners, fillers and other agents for modifying the flexural modulus of the matrix, and additives that affect the release rate, penetration and absorption of a bioactive agent, when present in the bioadhesive, as discussed in more detail hereinbelow.

Concentrations of the Ingredients:

According to some embodiments of the present invention, the bioadhesive comprises gelatin, alginate, MMT and a coupling agent, while each of these ingredients is present in the bioadhesive formulation at a concentration which results from combining the first formulation and the second formulation at a given ratio.

It is noted herein that for the bioadhesive formulation to be useful and effective, the concentration of its polymers is selected to afford a workable and pliable consistency, primarily in terms of viscosity. Therefore the high limit of the range of polymer concentration of workable formulations, and particularly that of gelatin, cannot exceed a certain value. Exceeding one or more of these maximal range values may result in an impracticable formulation that may not be formed or not be pliable for application.

The concentration of the various ingredients in any one of the first and second formulations is therefore set accordion to the volume or weight ratio at which the first and second formulations are combined. Since the formulations are typically made as liquids, it is more practical to refer to the combination of the first and second formulations in terms of volume, however it is noted that the combination of the first and second formulations can also be referred to in terms of weight.

According to some embodiments, combining the first formulation and the second formulation is effected at volume ratio of 1:9, 2:8 (1:4), 3:7, 4:6, 5:5 (1:1), 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1 or 25:1 parts of the first formulation and the second formulation respectively, or any ratio between 1:9 to 25:1. In some embodiments, the volume ratio ranges from 1:1 to 25:1 parts of the first formulation and the second formulation respectively. In some embodiments, the volume ratio ranges from 1:1 to 1:10 parts of the first formulation and the second formulation respectively. In some embodiments, the volume ratio ranges from 2:1 to 6:1 parts of the first formulation and the second formulation respectively. In some embodiments, the volume ratio is 4:1 parts of the first formulation and the second formulation.

According to some embodiments of the present invention, the concentration of gelatin in a bioadhesive formulation obtained by combining the first formulation and the second formulation at volume ratio of 1:9 to 25:1, is 500 mg/ml or lower. According to some embodiments of the present invention, the concentration of gelatin in the bioadhesive formulation ranges from 50 mg/ml to 500 mg/ml. In some embodiments, the gelatin content in the bioadhesive formulation ranges from 100 mg/ml to 500 mg/ml, from 100 mg/ml to 400 mg/ml, from 100 mg/ml to 300 mg/ml, from 100 mg/ml to 200 mg/ml, or from 50 mg/ml to 400 mg/ml, from 50 mg/ml to 300 mg/ml, from 50 mg/ml to 200 mg/ml or from 50 mg/ml to 100 mg/ml, including any value between the above-indicated ranges.

According to some embodiments of the present invention, the concentration of alginate in a bioadhesive formulation obtained by combining the first formulation and the second formulation at volume ratio of 1:9 to 25:1, ranges from 5 mg/ml to 100 mg/ml. In some embodiments, the alginate content in the bioadhesive formulation ranges from 10 mg/ml to 100 mg/ml, from 10 mg/ml to 90 mg/ml, from 10 mg/ml to 80 mg/ml, from 10 mg/ml to 70 mg/ml, from 10 mg/ml to 60 mg/ml, from 10 mg/ml to 50 mg/ml, from 10 mg/ml to 40 mg/ml, or from 5 mg/ml to 90 mg/ml, from 5 mg/ml to 80 mg/ml, from 5 mg/ml to 70 mg/ml, from 5 mg/ml to 60 mg/ml, from 5 mg/ml to 50 mg/ml, from 5 mg/ml to 40 mg/ml, from 5 mg/ml to 30 mg/ml, from 5 mg/ml to 20 mg/ml, or from 5 mg/ml to 10 mg/ml, including any value between the above-indicated ranges.

According to some embodiments of the present invention, the concentration of montmorillonite in a bioadhesive formulation obtained by combining the first formulation and the second formulation at volume ratio of 1:9 to 25:1, ranges from 1 mg/ml to 100 mg/ml. In some embodiments, the montmorillonite content in the bioadhesive formulation ranges from 1 mg/ml to 100 mg/ml, from 1 mg/ml to 90 mg/ml, from 1 mg/ml to 80 mg/ml, from 1 mg/ml to 70 mg/ml, from 1 mg/ml to 60 mg/ml, from 1 mg/ml to 50 mg/ml, from 1 mg/ml to 40 mg/ml, from 1 mg/ml to 30 mg/ml, from 1 mg/ml to 20 mg/ml, or from 1 mg/ml to 10 mg/ml, including any value between the above-indicated ranges.

According to some embodiments of the present invention, the concentration of the coupling agent in a bioadhesive formulation obtained by combining the first formulation and the second formulation at volume ratio of 1:9 to 25:1, ranges from 1 mg/ml to 50 mg/ml, from 1 mg/ml to 40 mg/ml, from 1 mg/ml to 30 mg/ml, from 1 mg/ml to 20 mg/ml, or from 1 mg/ml to 10 mg/ml, including any value between the above-indicated ranges.

As discussed hereinabove, since some coupling agents are known as cytotoxic or as generating cytotoxic moieties, it is desired to use relatively low amount of the coupling agent within a bioadhesive formulation. On the other hand, reducing the amount of a coupling agent in the formulation may result in reducing the bonding strength of the bioadhesive matrix formed from the formulation. The present inventors have demonstrated that bioadhesives containing 10-40 mg/ml or even lower amounts of a coupling agent can be used to provide bioadhesive matrices in which the bonding strength is not compromised substantially.

The concentration of some of the ingredients of the bioadhesive presented herein are summarized in the table below.

Bioadhesive ingredient concentrations

| | | | | |
|---|---|---|---|---|
| Gelatin | alginate | MMT | EDC | Water |

Concentration in mg/ml

| | | | | | |
|---|---|---|---|---|---|
| Min | 50 | 5 | 1 | 1 | 1000 |
| Max | 500 | 100 | 100 | 50 | 1000 |
| Example | 400 | 10 | 20 | 20 | 1000 |

Concentration in %

| | | | | | |
|---|---|---|---|---|---|
| Min | 4.7 | 0.5 | 0.1 | 0.1 | 94.2 |
| Max | 28.6 | 5.7 | 5.7 | 2.9 | 57.1 |
| Example | 27.6 | 0.7 | 1.4 | 1.4 | 69.0 |

A few exemplary bioadhesive formulations are presented below, each comprising gelatin, alginate, montmorillonite and a coupling agent at the indicated concentrations, given as percent by weight per volume of the total volume of a bioadhesive afforded by combining the first formulation and the second formulation at volume ratio of 1:9 to 25:1:

Exemplary bioadhesive formulation I: 200 mg/ml gelatin, 10 mg/ml alginate, 5 mg/ml montmorillonite and 10 mg/ml EDC as a carbodiimide-type coupling agent;

Exemplary bioadhesive formulation II: 300 mg/ml gelatin, 10 mg/ml alginate, 5 mg/ml montmorillonite and 10 mg/ml EDC;

Exemplary bioadhesive formulation III: 400 mg/ml gelatin, 10 mg/ml alginate, 5 mg/ml montmorillonite and 10 mg/ml EDC;

Exemplary bioadhesive formulation IV: 300 mg/ml gelatin, 20 mg/ml alginate, 5 mg/ml montmorillonite and 15 mg/ml EDC;

Exemplary bioadhesive formulation V: 300 mg/ml gelatin, 20 mg/ml alginate, 5 mg/ml montmorillonite and 15 mg/ml EDC;

Exemplary bioadhesive formulation VI: 300 mg/ml gelatin, 20 mg/ml alginate, 5 mg/ml montmorillonite and 15 mg/ml EDC;

Exemplary bioadhesive formulation VII: 400 mg/ml gelatin, 20 mg/ml alginate, 10 mg/ml montmorillonite and 20 mg/ml EDC;

Exemplary bioadhesive formulation VIII: 400 mg/ml gelatin, 30 mg/ml alginate, 15 mg/ml montmorillonite and 20 mg/ml EDC;

Exemplary bioadhesive formulation IX: 400 mg/ml gelatin, 40 mg/ml alginate, 20 mg/ml montmorillonite and 20 mg/ml EDC; or

Exemplary bioadhesive formulation X: 400 mg/ml gelatin, 10 mg/ml alginate, 10 mg/ml montmorillonite and 15 mg/ml EDC; or

Exemplary bioadhesive formulation XI: 400 mg/ml gelatin, 10 mg/ml alginate, 20 mg/ml montmorillonite and 15 mg/ml EDC.

It is noted herein that, in some embodiments of the present invention, the concentration of the coupling agent may be lowered by the presence of an additive, without compromising the formulation's performance.

It is noted herein that, in some embodiments of the present invention, the formulation may include an optional additive, as presented hereinabove, for improving the performance of the bioadhesive and rendering it more suitable for use in a wider range of applications.

Any of the aforementioned exemplary formulation may further include one or more bioactive agents as described herein.

According to embodiments of the present invention, the solvent of the first and second formulations is water. In some embodiments, the water content in the combined bioadhesive formulation afforded by combining the first and second formulations at volume ratio of 1:9 to 25:1 ranges from 40% to 95% of the bioadhesive formulation.

It is noted herein that other combinations of component concentrations are contemplated, some of which have been demonstrated in the Examples section that follows.

A Kit for Forming a Bioadhesive:

The bioadhesive presented herein, also referred to herein as a sealant, comprising gelatin, alginate and MMT, is cured upon contacting a coupling agent. Thus an effective mean to store, form and dispense the bioadhesive is a kit in which the coupling agent is kept separated from the gelatin and alginate.

According to an aspect of some embodiments of the present invention, there is provided a kit for storing, forming and/or applying a bioadhesive; the kit includes at least two containers, referred to herein as a first container and a second container, wherein the first container contains a first formulation, and the second container contains a second formulation. In some embodiments, the first formulation includes gelatin and alginate, and the second formulation includes a coupling agent for coupling the gelatin and/or for coupling the alginate and/or for coupling the gelatin to the alginate, and the clay mineral montmorillonite (MMT) is included in any one of the first formulation and the second formulation. As long as the two containers are kept sealed and under acceptable storage conditions, and the first and second formulations are not mixed, the bioadhesive will not cure or disintegrate.

It is noted herein that the abovementioned optional additives, as well as the optional bioactive agents described herein, may be added to any one of the first and/or second formulations.

In some embodiments, the kit includes at least two containers, each containing the constituents corresponding to a particular formulation, which have been pre-dissolved in a solvent to a specific concentration such that mixing the two formulations results in the bioadhesive. Additionally or alternatively, the kit includes one or more compartments, each containing a pre-measured amount of a dry powder of one or more constituent of the bioadhesive formulation, and a separate compartment containing a pre-measured amount of the solvent, such that mixing the powder(s) and the solvent results in the bioadhesive.

The kit may further include mixing and stirring tools, bowls, applicators, freshness indicators, tamper-proof measures and printed matter for instructions for the user.

An Applicator Device:

The kit may be in the form, or include a device, an applicator or a dispenser for expelling, dispensing and applying measured amounts of each of the first and second formulations controllably and optionally synchronously, each of which is dispensed from the individual container serving as a cartridge of the individual formulation.

Hence, according to another aspect of some embodiments of the present invention, there is provided an integrated dual chamber dispenser or applicator device for use in forming, dispensing and applying the bioadhesive presented herein.

According to some embodiments of the present invention, the applicator device includes a dual barrel cartridge assembly with a joint delivery port with a mount therein for coupling with a mixing tube. Integrated applicators suitable for applying the bioadhesive formulation presented herein, may follow the design of any applicator for two-part chemistry adhesives which require efficient dispensing under controlled and safe conditions of two sub-formulations from separate compartments.

For example, the applicator device includes the first container in a form of a first syringe having a first barrel defining a first chamber for retaining the first formulation, and a first plunger having one end received in the chamber for extruding the first formulation from the first chamber;

the second container in a form of a second syringe having a second barrel defining a second chamber for retaining the second formulation, and a second plunger having one end received in the second chamber extruding the second formulation from the second chamber;

a nozzle having a distal end, a proximal end, and a lumen extending through the nozzle and means for connecting the proximal end of the nozzle to the first chamber and the second chamber such that the first formulation and the second formulation come in contact in the lumen,

whereby forming the bioadhesive that may be ejected through the nozzle upon driving the first plunger and the second plunger.

Additional applicator devices, which can be used to mix, form, dispense and apply the bioadhesives presented herein, are disclosed, for example, in U.S. Pat. Nos. 4,044,757, 4,979,942, 5,082,147, 6,732,887, 7,530,808, 7,635,343, 7,699,803, 8,074,843, all of which are incorporated herein by reference as if full set forth herein.

Characteristics of the Bioadhesive Formulation:

As discussed hereinabove, a design of an effective bioadhesive should comply with several requirements, which include, for example, workability/usability and efficiency, both translated also into safety.

Under the workability and safety considerations, an effective pre-curing bioadhesive, referred to herein as a bioadhesive formulation, should exhibit a viscosity at room temperature which allows the practitioner to apply and use the bioadhesive under, e.g., clinic conditions. For example, a bioadhesive formulation which is too viscous and thus difficult to mix and not spreadable, would be difficult to apply on tissues, while on the other hand a bioadhesive formulation which is not viscous enough and is thus too fluid, would be runny and maybe accompanied by undesirable leakage, insufficient adhesion and overall, in enhanced adverse side effects and reduced safety.

Further under the workability and safety consideration, an effective bioadhesive should exhibit a curing time, as defined herein, which allows completing an operation utilizing the bioadhesive in a relatively short time, so as to avoid prolonged operations which may result in enhanced adverse side effects and reduced safety, yet, on the other hand, be sufficiently long to allow accurate positioning and optional re-positioning if required.

In some embodiments instantaneous or rapid bonding of objects may be desired, for example in cases where the objects are close to one another, relatively small, or easily positioned optimally such that a re-positioning step may not be required; hence, short curing time may be suitable in some embodiments of the present invention. In such cases it is desirable that the bioadhesive formulation cures in a relatively short period of time.

In some embodiments instantaneous bonding of objects may be undesired, for example in cases where the objects are not easily positioned optimally and a re-positioning step may be required; hence, exceedingly short curing time may be impractical in some embodiments of the present invention. In such cases it is desirable that the bioadhesive formulation allows a period of time for re-positioning (separation and re-adjoining) of the objects to be bonded to one-another.

This range of time (window) in which the bioadhesive cures is referred to as the workable time, as discussed further hereinbelow.

When referring to a viscosity of a multi-part bioadhesive formulation, such as the bioadhesive formulation that is afforded by combining the first and second formulations presented herein, it pertains essentially to the more viscous formulation, namely the formulation which contains gelatin, alginate or a mixture of gelatin and alginate with or without montmorillonite and/or any bioactive agent or other additives. This more viscous formulation will essentially maintain similar viscosity as long as the chemical composition thereof is maintained, namely the concentration of the viscosity-conferring polymers (or water content), temperature and molecular structure remains unchanged. Alternatively, a reference to viscosity pertains to the combined bioadhesive formulation comprising all ingredients, which is afforded shortly subsequent to mixing the first and second formulations. While the dissolved polymers contribute the most to the high viscosity, the addition of a coupling agent will change the chemical composition of the polymers by crosslinking, which irreversibly alters the viscosity of the bioadhesive, essentially regardless of temperature and water content. From practical considerations, when referring herein to viscosity, it is referred to the first formulation containing gelatin and alginate, while the second formulation containing the coupling agent is considered less viscous and minor in volume relative to the viscous part. Measuring the viscosity of the bioadhesive formulation using standard and common practices and equipment may be impractical due to the relatively short curing time. Gelatin and alginate are the predominant contributors to viscosity, while all other constituents and additives are minor viscosity modifiers. The outstanding constituents are the crosslinking agent and the crosslinking promoting agent, which have a direct effect on the viscosity of the formulation, as these agents are responsible for curing (hardening) the formulation to a solid bioadhesive matrix. Hence, when referring the viscosity of the combined formulation, one may refer to the viscosity of the first formulation. It is safely assumed that the viscosity of the first formulation, lacking the hardening agents is essentially the same as the viscosity of the combined first and second formulations.

As discussed hereinbelow, the bioadhesive formulation is characterized by a “workable time”, referring to the time period between the time point where all ingredients of the bioadhesive are mixed together, to the time point where the bioadhesive is too viscous to work-up. Hence, the reported viscosity of a bioadhesive formulation, according to some embodiments of the present invention, is the effective viscosity during the workable time.

Dynamic viscosity is quantified by various units, depending on the measuring method and other factors. In the context of the present embodiments, dynamic viscosity is referred to in units of Newton second per square meter (N s m⁻² or Pa-sec), wherein 1 Pa-sec is equivalent to 1 kilogram per meter second (kg m⁻¹ s⁻¹) and equivalent to 10 poise (P). For example, water at 20° C. are said to have dynamic viscosity of 1 mPa-sec (0.001 Pa-sec), blood at 37° C. is characterized by a viscosity of 3-4 mPa-sec, and honey at 20° C. by 10 Pa-sec.

Thus, according to some embodiments of the present invention, the bioadhesive formulations presented herein are characterized by at least one of:

a room temperature viscosity that ranges from 1 Pa-sec to 50 Pa-sec (referring to either the gelatin-alginate solution, before adding the coupling agent solution, or to the final bioadhesive formulation containing all components just after being mixed together); and

a curing time under physiological conditions that ranges from 5 seconds to 30 minutes.

While the criteria for dynamic viscosity is given in Pa-sec units and its values are derived from particular viscosity measurements at a given ambient temperature, it is noted herein that dynamic viscosity can be expressed by other units, and measured by various methods and techniques, all of which can be used to characterize any given bioadhesive formulation or part thereof. For example, while it is simpler to measure dynamic viscosity at room temperature, it may also be useful to report and consider also the dynamic viscosity of bioadhesive formulations at a temperature higher than the working temperature, since it is more efficient and practical to mix and prepare the formulation at higher temperatures, such as 50° C. Alternatively, it may be more informative to report and consider dynamic viscosity at a temperature lower that the standard room temperature since, for example, most operating rooms are kept at a standard temperature lower than room temperature; as well as reporting and considering the viscosity of a bioadhesive formulation at or near body temperature, where the formulations is intended to be applied and used.

Hence, according to some embodiments of the present invention, the dynamic viscosity of the bioadhesive formulation, or the dynamic viscosity of the formulation part containing gelatin and alginate, ranges from 1 Pa-sec to 50 Pa-sec at 20° C., and/or 0.5 Pa-sec to 25 Pa-sec at 37° C.

According to some embodiments of the present invention, the room temperature dynamic viscosity of the bioadhesive formulation, or the dynamic viscosity of the formulation part containing gelatin and alginate, ranges from 1 Pa-sec to 5 Pa-sec, from 5 Pa-sec to 10 Pa-sec, from 10 Pa-sec to 15 Pa-sec, from 15 Pa-sec to 20 Pa-sec, from 20 Pa-sec to 25 Pa-sec, from 25 Pa-sec to 30 Pa-sec, from 30 Pa-sec to 35 Pa-sec, from 35 Pa-sec to 40 Pa-sec, from 40 Pa-sec to 45 Pa-sec, or from 45 Pa-sec to 50 Pa-sec.

As used herein, the phrase “curing time” describes a time period during which the bioadhesive formulation forms a bioadhesive matrix, as described herein.

It is noted herein that while the bioadhesive formulation begins to cure upon contacting the coupling agent with one or both gelatin and/or alginate, this coupling and crosslinking reaction is not instantaneous across the entire bulk of the mass of the formulation. Accordingly, the term “curing time” is defined such that it encompasses the entire process of matrix formation at all its stages, including the “workable time” and the “bonding time”. The “workable time” is the time-window between the moment of mixing all the ingredients of the formulation together, to the moment at which the formulation's viscosity is too high, presumably due to its curing process, to allow working with the formulation, namely applying, spreading, positioning and re-positioning, as discussed hereinabove. In the context of the viscosity characteristic of the bioadhesive formulation presented hereinabove, the viscosity is relevant during the workable time, until crosslinking prevails and turns the formulation too viscous. The “bonding time” is defined as the time which elapses from the moment the formulation is applied on the object, to the moment at which the objects which are being bonded one to the other, are considered bonded at sufficient strength, so as to allow, for example, release of any fastening/tightening means (if used) and/or the continuation or completion of the procedure. The workable time and the bonding time may overlap to some extent, may continue one another respectively or may be discontinued, depending on the formulation, mode of its use as a single or multi-part formulation, the conditions of use and the objects' type and the bonding area.

According to some embodiments of the present invention, the workable time of the bioadhesive formulation presented herein, containing all ingredients mixed together, spans from 0 seconds to 30 minutes. Depending on the required and intended use of the formulation, it can be designed to exhibit various workable times which may span less than 5 seconds, less than 10 seconds or less than 30 seconds for applications not requiring long workable times. In some embodiments where longer workable times are required, the formulation is designed to exhibit a workable time of at least 30 seconds, at least 60 seconds, at least 120 seconds, at least 300 seconds, at least 600 seconds, at least 900 seconds, or at least 1800 seconds (30 minutes).

According to some embodiments of the present invention, the curing time of the bioadhesive formulation presented herein can be pre-determined to be rapid or slow, depending on the application of the bioadhesive. In general, the curing time ranges from 5 seconds to 30 minutes. Depending on the required and desired performance of the formulation, it can be designed to exhibit various curing times which may range, in case of rapid curing, from 5 to 20 seconds, from 5 to 30 seconds, or from 5 to 60 seconds. In case of slower curing, the curing time ranges from 30 to 60 seconds, from 60 to 120 seconds, from 30 to 300 seconds, from 60 to 600 seconds, or from 60 to 1800 seconds (30 minutes). Alternatively, in cases where the adhesion process is not restricted in time and other parameters such as strength and flexibility are more consequential, such as for example in topical (external) adhesion of a device to a patient's skin, the curing time may be longer than the aforementioned values, and can range from a few seconds to more than 30 minutes, and be, for example, 40 minutes, 50 minutes, 60 minutes, and even 120 minutes, including any intermediate value from 30 minutes and 120 minutes.

The Resulting Bioadhesive Matrix:

The bioadhesive matrix is a result of the curing process which takes place between some of the ingredients of the bioadhesive formulation, and hence the matrix comprises gelatin and alginate, coupled to one another, as discussed herein, and montmorillonite associated therewith (e.g., entrapped therein), and optionally other constituents of the formulation which became associated with the matrix (e.g., entrapped therein).

As discussed hereinabove, the bioadhesive formulation as described herein is designed to form a corresponding bioadhesive matrix, and is hence designed such that the bioadhesive matrix exhibits a desired performance.

According to some embodiments of the present invention, the bioadhesive described herein is designed such that upon its curing, it is characterized by a high bonding strength of viable biological objects as defined herein, an optimal flexural modulus under physiological conditions, and an optimal biodegradability rate.

As used herein, the expression “optimal” relates to the performance of the formulation and/or corresponding matrix at a desired application. It is noted in this regard that different applications may require different parameters for an optimal performance, which may typically depend on the type of objects to be sealed or bonded, the dimensions of the objects to be bonded or area to be sealed, the contact area, the conditions the nature of the procedure calling for adhesion and other object- and procedure-dependent parameters.

While bonding strength of various viable tissues and other live or inanimate objects is a highly desired characteristic of a bioadhesive, it is a non-trivial ability of the bioadhesive formulations presented herein to bond various objects, such as viable and non-viable tissues, bone, skin, metal, plastic and other natural and synthetic polymeric substances, under physiological conditions of mucus/plasma/blood wet environments.

According to some embodiments of the present invention, the bioadhesive described herein is designed such that upon its curing, its burst strength is sufficient to seal a ruptured tissue.

An example of a procedure for determining the burst strength of a bioadhesive is provided in the Examples section below. In some embodiments of the present invention, the burst strength of the bioadhesive can be expressed in terms of the maximal pressure required to rupture a plug made from the bioadhesive matrix, that seals a hole of 3.0 mm uniform diameter, wherein the hole is punched in a collagen sheet of a thickness that ranges from about 0.04 mm to about 0.1 mm. In some embodiments, the plug (seal) is in the form of a layer of the bioadhesive about 1 mm thick, which is afforded by applying about 0.5 ml of the bioadhesive over and around the abovementioned hole.

In some embodiments of the present invention, the burst strength of the bioadhesive can be determined according to Standard Test Method for Burst Strength of Surgical Sealants ASTM F2392-04.

In some embodiments of the present invention, the burst strength of the bioadhesive, is expressed in terms of maximal pressure that ranges from 350 mmHg to 650 mmHg.

The binding strength can be experimentally determined from the slope of a stress-strain curve created during tensile tests conducted on a sample of bonded objects, and expressed in units of force per unit area (Newton per square meter (N/m²) or dynes per square centimeter), namely Pascals (Pa), megaPascals (MPa) or gigaPascals (GPa).

The phrase “bonding strength” as used herein describes the maximum amount of tensile stress that a pair of bonded objects of given materials can be subjected to before they break apart.

According to some embodiments of the present invention, the bioadhesive matrix afforded from the bioadhesive formulation presented herein is characterized by a maximal bonding strength of viable biological objects that ranges from about 2,000 pascal (2 MPa) to about 60,000 pascal (60 MPa) at peak bonding. According to some embodiments, the maximal bonding strength of viable biological objects, exhibited by the bioadhesive matrix presented herein ranges from 2 MPa to 10 MPa, from 5 MPa to 20 MPa, from 15 MPa to 30 MPa, from 20 MPa to 40 MPa, from 30 MPa to 50 MPa, or from 40 MPa to 60 MPa.

Another desired characteristic of the bioadhesive matrix, according to some embodiments of the present invention, is the extent of its capacity to bend and flex under stress and pressure without breaking or detaching from the objects it bonds, while being under physiological conditions, especially wet and swelled by absorbing water from the environment.

Depending on the intended use and conditions, the bioadhesive matrix is expected to perform (maintain its adhesive role in physiological conditions) over time and under intermittent or continuous motion, stress, deformation, bending, stretching, pressure and tear. According to some embodiments of the present invention, the matrix is characterized by a flexural strength (modulus) under physiological conditions that ranges from 0.5 MPa to 200 MPa. Alternatively, the flexural modulus under physiological conditions, exhibited by the bioadhesive matrix presented herein, ranges from 0.5 MPa to 5 MPa, from 1 MPa to 10 MPa, from 5 MPa to 20 MPa, from 10 MPa to 15 MPa, from 15 MPa to 20 MPa, from 20 MPa to 30 MPa, from 30 MPa to 50 MPa, from 50 MPa to 100 MPa, from 75 MPa to 150 MPa, from 100 MPa to 150 MPa, or from 155 MPa to 200 MPa.

According to some embodiments of the present invention, the bioadhesive matrix presented herein is biodegradable.

In order to be used effectively in various internal or external medical procedures and particularly in internal surgical procedures, the bioadhesive matrix presented herein further exhibits an optimal biodegradation rate, allowing it to bond the objects for a sufficient length of time so as to exhibit its intended use before disintegrating.

The term “biodegradable” and any adjective, conjugation and declination thereof as used herein, refers to a characteristic of a material to undergo chemical and/or physical transformation from a detectable solid, semi-solid, gel, mucus or otherwise a localized form, to a delocalized and/or undetectable form such as any soluble, washable, volatile, absorbable and/or resorbable breakdown products or metabolites thereof. A biodegradable material undergoes such transformation at physiological conditions due to the action of chemical, biological and/or physical factors, such as, for example, innate chemical bond lability, enzymatic breakdown processes, melting, dissolution and any combination thereof.

Depending on the chemical and physical characteristics of the bioadhesive matrix and the location of its application, and the intended use thereof, the process of biodegradation of the matrix can span days to weeks to months. The phrase “biodegradability rate” is defined herein as the period of time between application of a bioadhesive formulation to the time by which the resulting bioadhesive matrix is no longer present as a bioadhesive matrix. By being “no longer present” it is meant that substance(s) that can be attributed to the original matrix can no longer be detected at the site of application of the bioadhesive formulation at a substantial level, or that traces thereof which may still be detected in the original site beyond normal levels can no longer bond tissue or linger at that site.

In general, the bonding strength of the cured bioadhesive presented herein, will start to degrade to some extent under physiological conditions. This degradation in strength is caused by the breakdown of the matrix, which is effected by a combination of factors, including chemical processes (swelling, dissolution and spontaneous chemical degradation), biological processes (enzymatically driven reactions, formation of new un-bonded cells and other tissue components and death of bonded cells and other tissue components), mechanical processes (stress, strain and tear) and the likes. For the sake of simplicity, the collection of factors and processes that degrade the bonding strength of the bioadhesives presented herein are encompassed and unified under the phrase “biodegradability rate”.

According to some embodiments of the present invention, the intended use of the bioadhesive is to hold biological objects attached to one-another for a time period long enough for the object to splice, fuse or heal. The period of time depends on the objects and on the medical procedure being performed. For example, the bioadhesive adjoins two edges of an incision strong and long enough to seal the incision, which is a form of a rupture, and allow the incision to repair itself and heal; the incision may be in an internal site in the body or on the surface (skin and muscle). In another example, one of the objects is a patch of skin and the other object is an inanimate medical device, in which case the bioadhesive is intended to hold the device affixed to the skin until it fulfills its purpose or until the bioadhesive is replaced.

The biodegradability of the bioadhesive can be manipulated by a combination of factors, starting at the composition, namely relative concentration of the polymers and the crosslinking density, additives that can alter the molecular structure of the bioadhesive (more and varied crosslinks), biodegradation accelerators/enhancers and biodegradation inhibitors/suppressors. Another factor that can be used to manipulate degradability is the macroscopic shape and structure of the bioadhesive, namely its surface area, accessibility to the surrounding medium, size of the treated area and the likes.

Hence, according to some embodiments of the present invention, the biodegradability rate of the bioadhesive presented herein ranges from about 7 days to about 6 months. In some cases the degradability period can be made shorter and range from 1 week to 1 month, including any time period in between, such as from 10 days to 3 weeks. In other cases, the degradability period can be made longer, e.g. 1-6 months, and range from 1 month to 2 months, from 2 month to 3 months or range from 2 months to 6 months.

Another parameter that can be used to determine the time factor involved in the bonding strength of any given bioadhesive, is the half time of bonding strength retention, namely the period of time by which the maximal bonding strength reaches half its value, T_{1/2}.

According to some embodiments of the present invention, T_{1/2} ranges from about 1 day to about 5 months and any value therebetween. For example, for short-adhesion period applications, T_{1/2} ranges from about 1 week to about two weeks, or from about 10 days to about 1 month. For longer adhesion periods, T_{1/2} ranges from about 1 month to about 2 months, or from about 2 months to about 3 months, or from about 3 months to about 4 months, or from about 3 months to about 5 months.

It should be noted herein that while a minimal bonding time and an optimal biodegradability rate are discussed, the bioadhesive presented herein can be removed before it is biodegraded, and its bonding time can be shortened intentionally by mechanical and chemical means.

Drug-Eluting Bioadhesive Formulations:

According to some embodiments of the present invention, a bioadhesive as described herein further comprises one or more bioactive agent(s). In some embodiments, such a formulation is designed to afford a drug-eluting bioadhesive upon curing. In other words, bioadhesives that include a bioactive agent, cure to form a drug-eluting bioadhesive in which the bioactive agent is incorporated. In some embodiments, such drug-eluting bioadhesives are formed such that the bioactive agent is released therefrom upon contacting a physiological medium. Thus, the bioadhesive, according to some embodiments of the present invention, can be used for various bioadhesion applications, as discussed herein, while at the same time serving as a reservoir and vehicle for delivering a bioactive agent.

It is noted herein that while the incorporation of a bioactive agent in the bioadhesive may affect the characteristics thereof, the bioadhesive is designed to possess desired properties presented hereinabove while adding the capacity of eluting bioactive agent(s) as discussed hereinbelow.

The term “incorporated”, as used in the context of a bioactive agent and a bioadhesive, according to some embodiments of the present invention, is used synonymously with terms such as “sequestered”, “loaded”, “encapsulated”, “associated with”, “charged” and any inflection of these terms, all of which are used interchangeably to describe the presence of the bioactive agent, as defined hereinbelow, within the bioadhesive. A sequestered bioactive agent can elute or be released from the bioadhesive via, for example, diffusion, dissolution, elution, extraction, leaching, as a result of any or combination of wetting, swelling, dissolution, chemical breakdown, degradation, biodegradation, enzymatic decomposition and other processes that affect the bioadhesive. A bioactive agent may also elute from the bioadhesive without any significant change to the bioadhesive structure, or with partial change.

As used herein, the phrase “bioactive agent” describes a molecule, compound, complex, adduct and/or composite that exerts one or more biological and/or pharmaceutical activities. The bioactive agent can thus be used, for example, to relieve pain, prevent inflammation, prevent and/or reduce and/or eradicate an infection, promote wound healing, promote tissue regeneration, effect tumor/metastasis eradication/suppression, effect local immune-system suppression, and/or to prevent, ameliorate or treat various medical conditions.

“Bioactive agents”, “pharmaceutically active agents”, “pharmaceutically active materials”, “pharmaceuticals”, “therapeutic active agents”, “biologically active agents”, “therapeutic agents”, “medicine”, “medicament”, “drugs” and other related terms may be used herein interchangeably, and all of which are meant to be encompassed by the term “bioactive agent”.

The term “bioactive agent” in the context of the present invention also includes diagnostic agents, including, for example, chromogenic, fluorescent, luminescent, phosphorescent agents used for marking, tracing, imaging and identifying various biological elements such as small and macromolecules, cells, tissue and organs; as well as radioactive materials which can serve for both radiotherapy and tracing, for destroying harmful tissues such as tumors/metastases in the local area, or to inhibit growth of healthy tissues, such as in current stent applications; or as biomarkers for use in nuclear medicine and radio-imaging.

Bioactive agents useful in accordance with the present invention may be used singly or in combination, namely more than one type of bioactive agents may be used together in one bioadhesive formulation, and therefore be released simultaneously from the bioadhesive.

In some embodiments, the concentration of a bioactive agent in the formulation ranges from 0.1 percent weight per volume to 10 percent weight per volume of the total volume of said formulation, and even more in some embodiments. Higher and lower values of the content of the bioactive agent ate also contemplated, depending on the nature of the bioactive agent used and the intended use of the bioadhesive.

When using the term “bioactive agent” in the context of releasing or eluting a bioactive agent, it is meant that the bioactive agent is substantially active upon its release.

As discussed hereinbelow, the bioactive agent may have an influence on the bioadhesive by virtue of its own reactivity with one or more of the bioadhesive components, or by virtue of its chemical and/or physical properties per-se. It is therefore noted that in general, the bioactive agent is selected suitable for being incorporated into the bioadhesive such that it can elute from the bioadhesive in the intended effective amount and release rate, while allowing the pre-curing bioadhesive formulation to exhibit desired properties, as discussed herein, and while allowing the bioadhesive to cure that exhibits the desired properties, as discussed herein. For example, any agent that interferes with the coupling and crosslinking reaction is excluded from the scope of the invention. For example, bioactive agents exhibiting a carboxylic group or a primary amine group may react with a coupling agent which is selected for its reactivity towards such functional groups. In such cases, in order to maintain desirable characteristics of the resulting matrix, some adjustments may be introduced to the bioadhesive formulation in terms of the type of ingredients and their concentrations.

A bioactive agent, according to some embodiments of the present invention, can be, for example, a macro-biomolecule or a small, organic molecule.

According to some embodiments of the present invention, the bioactive agent is a non-proteinous substance, namely a substance possessing no more than four amino acid residues in its structure.

According to some embodiments of the present invention, the bioactive agent is a non-carbohydrate substance, namely a substance possessing no more than four sugar (aminoglycoside inclusive) moieties in its structure.

According to some embodiments of the present invention, the bioactive agent is substantially devoid of one or more of the following functional groups: a carboxyl, a primary amine, a hydroxyl, a sulfhydroxyl and an aldehyde.

The term “macro-biomolecules” as used herein, refers to a polymeric biochemical substance, or biopolymers, that occur naturally in living organisms. Amino acids and nucleic acids are some of the most important building blocks of polymeric macro-biomolecules, therefore macro-biomolecules are typically comprised of one or more chains of polymerized amino acids, polymerized nucleic acids, polymerized saccharides, polymerized lipids and combinations thereof. Macromolecules may comprise a complex of several macromolecular subunits which may be covalently or non-covalently attached to one another. Hence, a ribosome, a cell organelle and even an intact virus can be regarded as a macro-biomolecule.

A macro-biomolecule, as used herein, has a molecular weight higher than 1000 dalton (Da), and can be higher than 3000 Da, higher than 5000 Da, higher than 10 kDa and even higher than 50 KDa.

Representative examples of macro-biomolecules, which can be beneficially incorporated in the bioadhesive described herein include, without limitation, peptides, polypeptides, proteins, enzymes, antibodies, oligonucleotides and labeled oligonucleotides, nucleic acid constructs, DNA, RNA, antisense, polysaccharides, viruses and any combination thereof, as well as cells, including intact cells or other sub-cellular components and cell fragments.

As used herein, the phrase “small organic molecule” or “small organic compound” refers to small compounds which consist primarily of carbon and hydrogen, along with nitrogen, oxygen, phosphorus and sulfur and other elements at a lower rate of occurrence. In the context of the present invention, the term “small” with respect to a compound, agent or molecule, refers to a molecular weight lower than about 1000 grams per mole. Hence, a small organic molecule has a molecular weight lower than 1000 Da, lower than 500 Da, lower than 300 Da, or lower than 100 Da.

Representative examples of small organic molecules, that can be beneficially incorporated in the bioadhesive described herein include, without limitation, angiogenesis-promoters, cytokines, chemokines, chemo-attractants, chemo-repellants, drugs, agonists, amino acids, antagonists, anti histamines, antibiotics, antigens, antidepressants, anti-hypertensive agents, analgesic and anesthetic agents, anti-inflammatory agents, antioxidants, anti-proliferative agents, immunosuppressive agents, clotting factors, osseointegration agents, anti-viral agents, chemotherapeutic agents, co-factors, fatty acids, growth factors, haptens, hormones, inhibitors, ligands, saccharides, radioisotopes, radiopharmaceuticals, steroids, toxins, vitamins, minerals and any combination thereof.

Representative examples of bioactive agents suitable for use in the context of the present embodiments include, without limitation, analgesic, anesthetic agents, antibiotics, antitumor and chemotherapy agents, agonists and antagonists agents, amino acids, angiogenesis-promoters, anorexics, antiallergics, antiarthritics, antiasthmatic agents, antibodies, anticholinergics, anticonvulsants, antidepressants, antidiabetic agents, antidiarrheals, antifungals, antigens, antihistamines, antihypertensive agents, antiinflammatory agents, antimigraine agents, antinauseants, antineoplastics, antioxidants, antiparkinsonism drugs, antiproliferative agents, antiprotozoans, antipruritics, antipsychotics, antipyretics, antisenses nucleic acid constructs, antispasmodics, antiviral agents, bile acids, calcium channel blockers, cardiovascular preparations, cells, central nervous system stimulants, chemo-attractants, chemokines, chemo-repellants, chemotherapeutic agents, cholesterol, co-factors, contraceptives, cytokines, decongestants, diuretics, DNA, Drugs and therapeutic agents, enzyme inhibitors, enzymes, fatty acids, glycolipids, growth factors, growth hormones, haemostatic and antihemorrhagic agents, haptens, hormone inhibitors, hormones, hypnotics, immunoactive agents, immunosuppressive agents, inhibitors and ligands, labeled oligonucleotides, microbicides, muscle relaxants, nucleic acid constructs, oligonucleotides, parasympatholytics, peptides, peripheral and cerebral vasodilators, phospholipids, polysaccharides, proteins, psychostimulants, radioisotopes, radiopharmaceuticals, receptor agonists, RNA, saccharides, saponins, sedatives, small organic molecules, spermicides, steroids, sympathomimetics, toxins, tranquilizers, vaccines, vasodilating agents, viral components, viral vectors, viruses, vitamins, and any combination thereof.

The bioactive agent may be selected to achieve either a local or a systemic response. The bioactive agent may be any prophylactic agent or therapeutic agent suitable for various topical, enteral and parenteral types of administration routes including, but not limited to sub- or trans-cutaneous, intradermal transdermal, transmucosal, intramuscular administration and mucosal administration.

One class of bioactive agents which can be encapsulated in the bioadhesive, according to some embodiments of the present invention, is the class of analgesic agents that alleviate pain e.g. NSAIDs, COX-2 inhibitors, opiates and morphinomimetics.

Another class of bioactive agents which can be incorporated in the bioadhesive, according to some embodiments of the present invention, is the class of anesthetic agents. Another class of bioactive agents which can be incorporated in the bioadhesive, according to some embodiments of the present invention, is the class of therapeutic agents that promote angiogenesis. Non-limiting examples include growth factors, cytokines, chemokines, steroids cell survival and proliferation agents.

Another class of bioactive agents which can be incorporated into the bioadhesive, according to some embodiments of the present invention, especially in certain embodiments wherein tissue regeneration is desirable, and application involving implantable devices and tissue healing, are cytokines, chemokines and related factors.

Non-limiting examples of immunosuppressive drugs or agents, commonly referred to herein as immunosupressants, include glucocorticoids, cytostatics, antibodies, drugs acting on immunophilins and other immunosupressants.

Non-limiting examples of haemostatic agents include kaolin, smectite and tranexamic acid.

It is noted herein that kaolin is an exemplary bioactive agent which has a limited solubility in the bioadhesive formulation, and is therefore added in the form of a dry powder, and thus acts, at least to some extent, also as a filler in the bioadhesive formulation. This dual function, bioactive agent and filler, may characterize any additive or bioactive agent which are encompassed by embodiments of the present invention and are contemplated therewith.

Additional bioactive agents which can be beneficially incorporated in the bioadhesive, according to some embodiments of the present invention, include cytotoxic factors or cell cycle inhibitors and other agents useful for interfering with cell proliferation.

Additional bioactive agents which can be beneficially incorporated into the bioadhesive, according to some embodiments of the present invention, include genetic therapeutic agents and proteins, such as ribozymes, anti-sense polynucelotides and polynucleotides coding for a specific product (including recombinant nucleic acids) such as genomic DNA, cDNA, or RNA. The polynucleotide can be provided in “naked” form or in connection with vector systems that enhances uptake and expression of polynucleotides. These can include DNA compacting agents (such as histones), non-infectious vectors (such as plasmids, lipids, liposomes, cationic polymers and cationic lipids) and viral vectors such as viruses and virus-like particles (i.e., synthetic particles made to act like viruses). The vector may further have attached peptide targeting sequences, anti-sense nucleic acids (DNA and RNA), and DNA chimeras which include gene sequences encoding for ferry proteins such as membrane translocating sequences (“MTS”), tRNA or rRNA to replace defective or deficient endogenous molecules and herpes simplex virus-1 (“VP22”).

Additional bioactive agents which can be beneficially incorporated in the bioadhesive, according to some embodiments of the present invention, include gene delivery agents, which may be either endogenously or exogenously controlled.

Additional bioactive agents which can be beneficially incorporated into the bioadhesive, according to some embodiments of the present invention, include the family of bone morphogenic proteins (“BMP's”) as dimers, homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA's encoding them.

Additional bioactive agents which can be beneficially incorporated into the bioadhesive, according to some embodiments of the present invention, include chemotherapeutic agents. Additional bioactive agents which can be beneficially incorporated into the bioadhesive, according to some embodiments of the present invention, include antibiotic agents.

Antiviral agents may include nucleoside phosphonates and other nucleoside analogs, AICAR (5-amino-4-imidazolecarboxamide ribonucleotide) analogs, glycolytic pathway inhibitors, glycerides, anionic polymers, and the like.

Additional bioactive agents which can be beneficially incorporated into the bioadhesive, according to some embodiments of the present invention, include viral and non-viral vectors.

Additional bioactive agents which can be beneficially incorporated into the bioadhesive, according to some embodiments of the present invention, include steroidal anti-inflammatory drugs. Additional bioactive agents which can be beneficially incorporated into the bioadhesive, according to some embodiments of the present invention, include anti-oxidants.

Additional bioactive agents which can be beneficially incorporated into the bioadhesive, according to some embodiments of the present invention, include vitamins.

Additional bioactive agents which can be beneficially incorporated into the bioadhesive, according to some embodiments of the present invention, include hormones.

Additional bioactive agents which can be beneficially incorporated into the bioadhesive, according to some embodiments of the present invention, include cells of human origin (autologous or allogeneic), including stem cells, or from an animal source (xenogeneic), which can be genetically engineered if desired to deliver proteins of interest.

Preparation of the Bioadhesive Formulation and Matrix Formation:

The bioadhesive presented herein, either containing or not containing a bioactive agent, are prepared by mixing all the ingredients together into a single concoction, at least in the sense of the formulation which is capable of curing. The single concoction can be formed ex vivo, in vitro or in situ, namely the formulation can be in the form of two or more sub-formulations kept separately, or as a set of dry powders and a pre-measured amount of solvent (water) kept separately, as discussed hereinbelow, which are combined to form the single concoction by one of the following manners.

In some embodiments, the bioadhesive is formed by contacting the first and second formulations. In some embodiments, the preparation of the bioadhesive further include mixing the first and second formulations.

In vitro means that the bioadhesive as a single concoction is formed by mixing (e.g., in a vial) all the components of the formulation, as these are defined, described and exemplified herein, prior to applying the formulation onto the object(s) to be bonded.

In situ means that the bioadhesive as a single concoction is formed by applying one of the first or second formulation on one object, and the other formulation on another object, and adjoining the objects together to form the single concoction at the site of adhesion; or by applying one of the first or second formulations on an object and thereafter applying the other formulation on the same object.

When applied to animated objects, in vitro corresponds to ex vivo, and in situ corresponds to in vivo.

In any eventuality, the formulation is kept under conditions where it is substantially unable to cure.

According to an aspect of some embodiments of the resent invention, there is provided a method of forming a bioadhesive matrix, which is effected by curing the bioadhesive formulation as described herein.

As used herein, the term “curing” includes an active procedure such as subjecting the formulation to certain conditions (e.g., heating and/or mixing, shear forces, etc.), as well as a passive procedure, which involved allowing the curing time to elapse.

In some embodiments, the method further comprises, prior to the curing, mixing the components of the formulation, namely, mixing the herein-described sub-formulations or mixing the herein-described powder(s) with the appropriate solvent(s).

As discussed hereinabove, the mixing can be effected ex vivo, in vivo, in vitro or in situ.

Use of the Bioadhesive:

In general, the bioadhesive presented herein can be used in the manufacturing of a product intended for adhering to and/or bonding objects, at least one of which is a biological object.

According to some embodiments of the present invention, the bioadhesives including a bioactive agent or not, and/or kits comprising the same, are identified for use in adhering a biological object. In some embodiments, the bioadhesive or kit is identified for use in sealing a rupture in a biological object. In some embodiments, the bioadhesive or kit is identified for use in bonding at least two objects to one another, wherein at least one of the objects is a biological object.

According to some embodiments of the present invention, the bioadhesive is used topically, namely the bioadhesive is used to adhere an object to the skin, to bond the edges of a lesion, to fix a skin graft, or seal a rupture in the skin.

Alternatively, the bioadhesive is used internally to adhere to internal organs and serve to adhere an object to an internal organ, to bond the edges of a lesion in an internal organ, to fix a graft to an internal organ, or seal a rupture in an internal organ.

For buccal applications, the bioadhesive, according to some embodiments of the present invention, adhere to the oral mucosa within seconds and remain adhered until fully eroded (biodegraded), without the need for a backing layer. The bioadhesive matrices presented herein combine high biocompatibility with flexibility in application.

For ocular applications, the bioadhesive, according to some embodiments of the present invention, adhere rapidly to the ocular mucosa and remain in place until fully eroded.

For intra-nasal applications, the bioadhesive, according to some embodiments of the present invention, adhere immediately to the nasal mucosa and remain in place until fully eroded. Drug-releasing bioadhesive matrices, according to some embodiments of the present invention, offer high drug loading capacity and nasal residence and release time to maximize drug efficacy.

For vaginal applications, the bioadhesive, according to some embodiments of the present invention, adhere to the vaginal mucosa within seconds and can remain adhered for several days until fully eroded. Drug-releasing bioadhesive matrices, according to some embodiments of the present invention, offer a safe effective administration and a desired systemic effect.

Thus, the phrase “biological object”, as used herein, refers to any viable/live part of an animal or plant, including a single live animal specimen. A live or viable biological object or tissue is defined as any major or minor part of a plant or animal that is still viable or alive and substantially kept in a physiological environment in order to stay viable or alive. Non-limiting examples of biological objects include any plant or animal, viable tissue samples, skin tissue, bone tissue, connective tissue, muscle tissue, nervous tissue and epithelial tissue. Also encompassed are edges of incisions made in an organ, such as skin, muscle, internal organ in any bodily site of an organism.

Inanimate objects are objects which cannot be revived, grafted, proliferate or otherwise show any signs of life as defined medically, and include objects of synthetic and/or biological origins. These include, for example, patches, bone-replacement parts, pace makers, ports and vents and any other medical device that required affixing and immobilization to a viable biological object as defined herein.

According to some embodiments of the present invention, inanimate biological objects can be made partially or entirely from animal or plant materials and products, or partially or entirely from synthetic substances. While the bioadhesive formulation is designed for use in or on viable biological objects, it is noted herein that is can be used effectively to bond biologic or synthetic inanimate objects like any adhesion agent or glue.

It is noted herein that the term “object” is meant to encompass one or more parts or portions of the same object, thus closing an incision by bonding the two sides of the incision in a tissue or an organ, by using the herein-described formulation, can be regarded as either bonding one object (the tissue or organ) or two objects (the two sides of the incision).

According to some embodiments, the drug-eluting matrix, resulting from a bioadhesive formulation which incorporates a bioactive agent, is used solely for its drug-eluting and drug-delivery faculties regardless of its bioadhesive faculty. Such a matrix can serve, for example, as a drug depot, and can be adhered to an organ or tissue where the release of the drug is beneficial (without bonding thereto another object).

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the formulation, composition, method, matrix or structure may include additional ingredients, steps and/or parts, but only if the additional ingredients, steps and/or parts do not materially alter the basic and novel characteristics of the claimed formulation, composition, method, matrix or structure.

As used herein, the singular form “a”, “an” and “the” include plural references unless the context clearly dictates otherwise. For example, the term “a compound” or “at least one compound” may include a plurality of compounds, including mixtures thereof. Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1

### Materials and Methods

Materials:

An exemplary basic bioadhesive matrix, according to some embodiments of the present invention, was prepared from an exemplary bioadhesive formulation containing some of the following ingredients:

Gelatin—In the example presented herein gelatin “type A” from porcine skin (90-110 bloom) was purchased from Sigma-Aldrich Cat#: G6144.

Alginate—In the example presented herein alginic acid sodium salt (viscosity about 250 cps, 2% (25° C.) was purchased from Sigma-Aldrich Cat#: A1112.

EDC—N-(3-Dimethylaminopropyl)-N′-ethylcarbodiimide hydrochloride was purchased from Sigma-Aldrich Cat#: E7750.

Kaolinite (Kaolin)—Al₂Si₂O₅(OH)₄, a well-known and widely used hemostatic agents, was purchased from Sigma-Aldrich Cat#: K1512.

Montmorillonite, sodium (Na⁺-MMT)—was purchased from Byk as CLOISITE-Na+.

General Applicator Equipment Included:

Double barrel syringes (Mixpac, SULZER);

Double Syringe, 2.5 ml, 4:1, PP—R natural 110865;

Plunger with Lip, 2.5 ml, 4:1, PE—HD natural 107714;

Static syringe mixer DN 2.5×8, 4:1/10:1, blue 112313;

BioGlue surgical adhesive, Cryolife. Lot: 14MGW062.

Methods:

An example of a bioadhesive (sealant), according to some embodiments of the present invention, was prepared as follows. Formulation A comprising 400 mg/ml gelatin and 10 mg/ml alginate was prepared by dissolving the substances in doubly-distilled water (DDW) while heating to 60° C. In formulations that contained a clay mineral (e.g., kaolin or MMT), the clay mineral was added to Formulation A before dissolving in alginate. Formulation B comprising 10, 15 or 20 mg/ml EDC was prepared using DDW as a carrier. The application of the bioadhesive, according to some embodiments of the present invention, was effected in all tests by combining Formulation A and Formulation B using double barrel syringe applicators.

The in-vitro burst strengths measurements of various bioadhesive matrices formed by curing various bioadhesive (sealant) formulations, according to some embodiments of the present invention, were tested using a custom-built mechanical burst tester following the Standard Test Method for Burst Strength of Surgical Sealants ASTM F2392-04 (2015). The principle of this test was to measure the maximal rupture pressure, expressed in mmHg units, at tissue leakage point that can be held by the cured bioadhesive. Briefly, a washed collagen sheet, cut from a commercially available collagen casing having a thickness that ranges from about 0.04 mm to about 0.1 mm (Fibran 51, Spain), was punctured by making a 3.0 mm uniform diameter hole therein, was used to mimic a tissue substrate. Approximately 0.5 ml of a test bioadhesive, according to some embodiments of the present invention, was applied to the collagen casing substrate, sealing the hole with a measured thickness of approximately 1 mm coating of the bioadhesive. Samples were placed onto a test unit 3-6 minutes after bioadhesive application and pressure was applied. The pressure at which the cured bioadhesive failure occurred was recorded using a digital manometer (Kobman SD1S6B70) as the maximal burst pressure. A minimum of 10 repetitions were carried out for each of the tested bioadhesives.

The swelling ratio and weight loss of various bioadhesives (sealants), according to some embodiments of the present invention, were tested by casting the uncured samples into 7.0×7.0×3.5 mm³ silicon molds using the double syringe applicator. After curing the bioadhesive samples were carefully removed and dried for 24 hours. The cured bioadhesive matrices were then weighed (W1) and immersed in 3 mL PBS (pH 7.0), placed in a static incubator at 37° C. and 100% relative humidity for 2, 6, and 24 hours. The cured bioadhesive samples were then weighed (W2) by removing the PBS and blotting using Kimwipes, dried for 24 hours and weighed again (W3). The swelling ratio and the weight loss were calculated based on 3-4 repetitions for each sample at each point of time according to the following equations:

Swelling ratio:(W2−W3)/W3×100% (Equation 1);

Weight loss:(W1−W3)/W3×100% (Equation 2).

### Example 2

### Burst Strength Test Results

The effect of gelatin and alginate concentrations on the burst strength in terms of mmHg of some embodiments of bioadhesives (sealants), crosslinked with 20 mg/ml EDC, according to some embodiments of the present invention, was tested according to the procedure described hereinabove, and the results are presented in Table 1.

TABLE 1

| Gelatin-Alginate sealants | | | | | | |
|---|---|---|---|---|---|---|
| crosslinked with 20 mg/ml | | Gelatin concentration [mg/ml] | | | | |
| EDC | | 200 | 300 | 400 | 500 | 600 |
| Alginate | 0 | 79.5 ± 27.9 | 236.3 ± 25.3 | 386.3 ± 63.2 | 455.3 ± 35.7 | 449.3 ± 148.4 |
| concentration | 10 | 133.1 ± 11.3 | 216.8 ± 32.9 | 351.0 ± 66.2 | 462.0 ± 63.0 | 496.5 ± 53.9 |
| [mg/ml] | 20 | 97.5 ± 16.8 | 204.0 ± 41.1 | 373.5 ± 78.0 | 427.5 ± 75.9 | |
| | 30 | 90.0 ± 15.0 | 231.0 ± 38.5 | 401.3 ± 68.7 | | |
| | 40 | 101.3 ± 12.8 | | | | |

The effect of EDC concentration on the burst strength (in terms of mmHg) of some embodiments of bioadhesives comprising gelatin and alginate, according to some embodiments of the present invention, was tested according to the procedure described hereinabove, and the results are presented in Table 2.

TABLE 2

| | | Gelatin-Alginate concentrations [mg/ml] | | |
|---|---|---|---|---|
| EDC effect | | 400-0 | 400-10 | 500 |
| EDC concentration | 10 | 330.0 ± 49.9 | 225.8 ± 49.8 | 360.8 ± 47.9 |
| [mg/ml] | 15 | 390.0 ± 78.8 | 358.5 ± 52.3 | 432.0 ± 74.4 |
| | 20 | 370.5 ± 67.8 | 408.0 ± 83.1 | 404.3 ± 29.0 |

The effect of kaolin concentration on the burst strength (in terms of mmHg) of an example of a bioadhesive (sealant) comprising 400 mg/ml gelatin, 10 mg/ml alginate, crosslinked with 15 mg/ml EDC, was tested according to the procedure described hereinabove, and the results are presented in Table 3.

TABLE 3
Kaolin concentration [mg/ml]
Added to a 400-10-15 Gelatin-Alginate-EDC formulation

| | 0 | 5 | 10 | 20 | 50 |
|---|---|---|---|---|---|
| Burst | 282.8 ± | 302.3 ± | 318.8 ± | 300.8 ± 118.0 | 390.0 ± 56.7 |
| strength | 55.6 | 115.0 | 68.6 | | |
| [mmHg] | | | | | |

The effect of montmorillonite (Na⁺-MMT) concentration on the burst strength (in terms of mmHg) of an example of a bioadhesive comprising 400 mg/ml gelatin, 10 mg/ml alginate, crosslinked with 15 mg/ml EDC, was tested according to the procedure described hereinabove, and the results are presented in Table 4.

TABLE 4
Na+-MMT concentration [mg/ml]
added to a 400-10-15 Gelatin-Alginate-EDC formulation

| | 0 | 2.5 | 5 | 10 | 15 | 20 |
|---|---|---|---|---|---|---|
| Burst strength | 282.8 ± 55.6 | 324.8 ± 23.7 | 404.3 ± 100.7 | 498.4 ± 51.0 | 486.0 ± 51.5 | 588.0 ± 83.1 |
| [mmHg] | | | | | | |

Examples of bioadhesives, according to some embodiments of the present invention, comprising 400 mg/ml gelatin, 10 mg/ml alginate, and crosslinked with 15 mg/ml EDC, and further comprising a clay mineral (kaolin or MMT), were compared according to their burst strength, and the results are presented in Table 5.

TABLE 5
Selected formulations based on the
400-10-15 (gelatin-alginate-EDC) formulation

| | No clay | Ref. + 5% | Ref. + 1% | Ref. + 2% |
|---|---|---|---|---|
| | additive (Ref.) | Kaolin | MMT | MMT |
| Burst | 282.8 ± 55.6 | 390.0 ± 560.7 | 498.4 ± 51.0 | 588.0 ± 83.1 |
| strength | | | | |
| [mmHg] | | | | |

As can be seen in Tables 1-5, increasing the concentration of gelatin resulted in higher burst strength of the cured bioadhesive matrix when crosslinked with 20 mg/ml EDC, while this effect was not observed when gelatin concentration was higher than 400 mg/ml. In addition, increasing the concentration of gelatin restricted the amount of alginate which can be added to the bioadhesive before it becomes too viscus. Accordingly, the viscosity of formulation containing 600 mg/ml (or more) gelatin was too high for proper handling with the double syringe applicator.

As can further be seen in Tables 1-5, alginate concentration showed minor effect on the burst strength of most tested bioadhesives, and crosslinking of gelatin-alginate bioadhesives with 15 mg/ml practically, according to some embodiments of the present invention, resulted in burst strength values that were similar to those obtained with 20 mg/ml EDC.

One of the more interesting observations stemming from the results presented in Tables 1-5 concern the addition of a clay mineral to the bioadhesives, according to some embodiments of the present invention. The incorporation of 5% kaolin to the 400-10-15 bioadhesives increased the burst strength of the cured samples by approximately 35% compared to a comparable clay-free bioadhesive. However, the burst strength of the 400-10-15 bioadhesives was increased in approximately 40% when only 0.5% MMT was added thereto. An increase of approximately 105% was obtained when 2% MMT was incorporated to the bioadhesives, according to some embodiments of the present invention.

Table 6 summarizes the comparative burst strength studies presented above.

TABLE 6

Burst strength [in mmHg] of
formulation containing 400 mg/ml
gelatin, 10 mg/ml alginate
and 15 mg/ml EDC

| Clay mineral content | Kaolin | MMT |
|---|---|---|
| (mg/ml) | formulations | formulations |
| 0 | 282.8 ± 55.6 | 282.8 ± 55.6 |
| 5 | 302.3 ± 115.0 | 404.3 ± 100.7 |
| 10 | 318.8 ± 68.6 | 498.4 ± 51.0 |
| 20 | 300.8 ± 118.0 | 588.0 ± 83.1 |

As can be seen in Table 6, unexpectedly, montmorillonite has been found to be a superior clay mineral additive for the bioadhesives, according to some embodiments of the present invention, compared to the more known and more widely used clay mineral kaolin, by conferring a higher burst strength to the bioadhesive formulation than kaolin.

### Example 3

### Swelling and Weight-Loss Test Results

The effect of montmorillonite concentrations on the swelling ratio of bioadhesives (sealants) comprising 400 mg/ml gelatin, 10 mg/ml alginate, crosslinked with 15 mg/ml EDC, according to some embodiments of the present invention, was tested according to the procedure described hereinabove, and the results are presented in Table 7.

TABLE 7
Montmorillonite concentration [% w/v]
added to a 400-10-15 Gelatin-Alginate-EDC formulation

| | | 400-10-15 | 0.25 | 0.5 | 1 | 1.5 | 2 |
|---|---|---|---|---|---|---|---|
| % | 2 h | 221.0 ± 14.7 | 234.3 ± 22.8 | 202.4 ± 25.2 | 209.6 ± 7.3 | 196.7 ± 19.9 | 195.0 ± 8.0 |
| Swelling | 6 h | 400.2 ± 16.4 | 393.5 ± 14.5 | 342.8 ± 20.2 | 356.7 ± 21.6 | 343.9 ± 18.0 | 323.1 ± 21.4 |
| ratio | 24 h | 667.0 ± 17.9 | 627.8 ± 43.1 | 589.9 ± 27.1 | 578.7 ± 18.1 | 530.8 ± 60.7 | 502.6 ± 19.6 |

The effect of montmorillonite concentrations on the weight loss of bioadhesives comprising 400 mg/ml gelatin, 10 mg/ml alginate, crosslinked with 15 mg/ml EDC, according to some embodiments of the present invention, was tested according to the procedure described hereinabove, and the results are presented in Table 8.

TABLE 8
Montmorillonite concentration [% w/v] added to a 400-10-15 Gelatin-Alginate-EDC formulation

| | | 400-10-15 | 0.25 | 0.5 | 1 | 1.5 | 2 |
|---|---|---|---|---|---|---|---|
| % Weight loss | 2 h | 3.7 ± 0.2 | 3.7 ± 0.5 | 3.9 ± 0.3 | 4.1 ± 0.3 | 3.6 ± 0.4 | 3.5 ± 0.1 |
| | 6 h | 3.7 ± 0.4 | 5.6 ± 0.1 | 4.8 ± 0.3 | 5.8 ± 0.4 | 5.8 ± 0.3 | 6.4 ± 0.9 |
| | 24 h | 9.3 ± 1.4 | 9.1 ± 1.7 | 5.8 ± 0.8 | 9.0 ± 1.0 | 8.3 ± 0.6 | 6.8 ± 0.6 |

The effect of kaolin concentrations on the swelling ratio of bioadhesives comprising 400 mg/ml gelatin, 10 mg/ml alginate, crosslinked with 15 mg/ml EDC, according to some embodiments of the present invention, was tested according to the procedure described hereinabove, and the results are presented in Table 9.

TABLE 9
Kaolin concentration [% w/v]
added to a 400-10-15 Gelatin-Alginate-EDC formulation

| | | 400-10-15 | 0.5 | 1 | 2 | 5 |
|---|---|---|---|---|---|---|
| % | 2 h | 221.0 ± 14.7 | 208.2 ± 23.5 | 207.4 ± 28.3 | 208.4 ± 25.7 | 271.4 ± 27.9 |
| Swelling | 6 h | 400.2 ± 16.4 | 365.4 ± 9.2 | 371.4 ± 11.3 | 376.4 ± 15.9 | |
| ratio | 24 h | 667.0 ± 17.9 | 591.1 ± 27.3 | 597.2 ± 11.8 | 598.9 ± 23.6 | |

The effect of kaolin concentrations on the weight loss of bioadhesives comprising 400 mg/ml gelatin, 10 mg/ml alginate, crosslinked with 15 mg/ml EDC, according to some embodiments of the present invention, was tested according to the procedure described hereinabove, and the results are presented in Table 10.

TABLE 10
Kaolin concentration [% w/v]
added to a 400-10-15 mg/ml Gelatin-Alginate-EDC formulation

| | | 400-10-15 | 0.5 | 1 | 2 | 5 |
|---|---|---|---|---|---|---|
| % | 2 hours | 3.7 ± 0.2 | 4.1 ± 0.3 | 3.6 ± 0.4 | 3.7 ± 0.7 | 7.1 ± 1.9 |
| Weight | 6 hours | 3.7 ± 0.4 | 3.9 ± 0.1 | 3.6 ± 0.7 | 4.4 ± 0.2 | |
| loss | 24 hours | 9.3 ± 1.4 | 6.0 ± 0.5 | 5.7 ± 0.2 | 7.0 ± 0.6 | |

As can be seen in Tables 7-10, an increase in montmorillonite concentration in the bioadhesive resulted in lower swelling ratio, with more significant differences found after 6 hours of incubation. The concentration of the clay mineral montmorillonite in the bioadhesives had a minor effect on the weight loss of the resulting sealant matrix.

Incorporation of 50 mg/ml kaolin into the 400-10-15 bioadhesives caused dissolution of the cured bioadhesive sample after less than 6 hours of incubation.

In general, it can be seen that the effect of incorporating clay minerals in the bioadhesive had a relatively small effect on water uptake (swelling) and weight loss of the resulting bioadhesive matrix.

An interesting observation stemming from the data presented in Tables 7-10, is the unexpected result of montmorillonite being a superior clay mineral for the bioadhesives, according to some embodiments of the present invention, compared to kaolin, by conferring a more stable and consistent bioadhesive matrix structure integrity, in terms of swelling and weight loss, when exposed to its working environment.

### Example 4

### Curing (Gelation) Time

Used as sealants, the bioadhesives presented herein are required, in some applications, to seal raptured tissues as fast as possible such as in life threatening bleeding situations. For such applications, the bioadhesive is required to exhibit a short curing (gelation) time. Viscosity of the bioadhesive formulation prior to final gelation is also a property to be monitored in certain applications.

As gelation time and viscosity are correlated to the relative concentrations of the main ingredients, experiments were conducted to determine the relations between concentration and gelation/viscosity.

Table 11 presents the results of the study which tested the effect of the concentration of the major ingredients of the bioadhesive on curing (gelation) time.

TABLE 11

| | mg/ml of Gelatin-Alginate- | |
|---|---|---|
| Variable | EDC + % Clay | Curing time (sec) |
| EDC/Alginate | 400-0-10 | 9.3 |
| | 400-0-15 | 8.0 |
| | 400-0-20 | 5.3 |
| | 400-10-10 | 8.7 |
| | 400-10-15 | 6.7 |
| | 400-10-20 | 5.0 |
| Gelatin/EDC | 200-0-15 | 14.3 |
| | 300-0-15 | 9.7 |
| | 400-0-15 | 8.0 |
| | 500-0-15 | 6.0 |
| | 200-10-15 | 12.7 |
| | 300-10-15 | 8.7 |
| | 400-10-15 | 6.7 |
| | 500-10-15 | 5.0 |
| Alginate | 400-0-15 | 8.0 |
| | 400-10-15 | 6.7 |
| | 400-20-15 | 6.3 |
| | 400-30-15 | 5.3 |
| MMT | 400-10-15 + 0.5% | 6.7 |
| | 400-10-15 + 1% | 6.0 |
| | 400-10-15 + 2% | 5.0 |
| Kaolin | 400-10-15 + 0.5% | 7.3 |
| | 400-10-15 + 2% | 7.3 |
| | 400-10-15 + 5% K | 6.7 |

As can be seen in Table 11, MMT has the curing acceleration effect similar to that of alginate and EDC, which take part in the crosslinking reaction, while kaolin does not show the same effect.

Table 12 presents the results of the study which tested the effect of the concentration of the major ingredients, without a coupling agent, on the viscosity (in Pascal sec) of the bioadhesive formulation prior to curing.

TABLE 12

| | 400-10 | | | | | | |
|---|---|---|---|---|---|---|---|
| Ingredient (mg/ml) | Gel-Alg | 5 Kaolin | 20 Kaolin | 50 Kaolin | 5 MMT | 10 MMT | 50 Kaolin |
| Viscosity [pa*s] | 1.1 | 0.8 | 1.2 | 1.4 | 2.0 | 2.8 | 6.2 |

### Example 5

### Coldwater Fish Skin Gelatin

Another exemplary bioadhesive matrix, according to some embodiments of the present invention, was prepared using coldwater fish skin gelatin.

### Materials and Methods

### Materials:

Coldwater fish skin “type A” gelatin (G7041), porcine skin “type A” gelatin (G6144), alginic acid sodium salt (A1112,) N-(3-dimethylaminopropyl)-N-ethylcarbodiimide hydrochloride (EDC) and kaolin (K1512) were purchased from Sigma-Aldrich, Rehovot, Israel. Sodium montmorillonite (Cloisite Na+) was purchased from BYK (USA).

### Preparation of Hemostatic Agent-Loaded Surgical Bioadhesives:

Preparation of the bioadhesives was based on dissolving various amounts of gelatin and alginate (Gel-Al) and hemostatic agent powders (kaolin or MMT) in distilled water, under heating up to 60° C. The crosslinking agent (EDC) was added to the Gel-Al solution containing the hemostatic agents just prior to the bioadhesive's use. Gelatin and alginate were characterized at concentrations of 200-600 and 0-40 mg/mL, respectively. The effect of kaolin and MMT was studied in concentrations of 5, 10, 15, 20, 50 and 2.5, 5, 10, 15, 20 mg/mL, respectively. The formulations are presented in the form of Gel-Al-EDC, where Gel is the concentration of gelatin, Al is the concentration of alginate, EDC is the concentration of the carbodiimide crosslinking agent (all in mg/mL).

In all experiments, the bioadhesive was applied using a double-syringe with a static mixer at a 4:1 volume ratio (Mixpac L-System, Sulzer, Switzerland) which provides consistent mixing of the polymer and crosslinker solutions.

The polymer solution containing fish gelatin was placed at room temperature, 25±2° C., for approximately ten minutes, thus allowing it to reach room temperature prior to application. The polymer solution containing porcine gelatin was used immediately after removal from the water bath, while still warm. The EDC concentration was 20 mg/mL. This concentration was found to be at a sufficient ratio for the polymers and to have low cytotoxicity.

### Evaluation of the Bioadhesive's Mechanical Properties:

Three types of mechanical tests were selected based on relevant standards for evaluation of the mechanical properties of the novel bioadhesives. The combination of these three methods enables establishing a thorough understanding of the bioadhesive function.

### Burst Strength Measurements:

The burst strength was tested using a custom-built mechanical burst device following the standard test method for Burst Strength of Surgical Sealants ASTM F2392-04. The principle of this test is to measure the maximal pressure at the tissue leakage point that can be held by the bioadhesive. A collagen casing (51 Fibran, Spain) with a uniform 3.0 mm diameter hole was used as the tissue substrate. Approximately 0.5 mL of bioadhesive were applied to the collagen casing substrate, sealing the defect with a measured thickness of approximately 1 mm. The sample was placed in the test unit and pressure was applied. The pressure at which bioadhesive failure occurred was recorded as the maximal burst pressure. A minimum of 10 repetitions were carried out for each formulation.

### Lap Shear Bonding Strength:

Adhesive bonding strength in lap shear was assessed according to ASTM F2255-05 using a 5500 Instron Universal Testing Machine (Instron Engineering Corp.) in order to investigate the mechanical properties under shear forces that are routinely applied to the skin in tissue adhesive applications. Briefly, sheets of collagen casing were cut into 2.5 cm wide strips. A one-centimeter area at the end of each strip was marked to be the overlapping area and the other end was folded in order to create a thick area that would be easy to grasp. Application of bioadhesive was executed in two different ways, due to the increase in the viscosity of the different solutions with the addition of more components:

Manual mixing was used for formulations that included only gelatin and EDC. 25 μL of polymer solution was spread on the marked area of each of two strips and 12.5 μL of crosslinking solution were added to one strip and were spread as evenly as possible. The two strips were overlapped immediately after application of the adhesive.

A double-syringe was used for the more viscous formulations, which contained alginate. An approximate amount of 60 μL bioadhesive was applied to one strip and the second strip was immediately set over this area.

A force of 1.2 N was applied to the bond area immediately after the overlapping, for a duration of 15 min, allowing the adhesive to cure and set. The entire procedure was carried out at room temperature, 25±2° C. The test specimens were placed in the grips of the testing machine so that the applied load coincided with the long axis of the specimen. The specimen was loaded to failure at a constant cross-head speed of 5 mm/min. Ten specimens were tested for each formulation; for each sample, the maximum force at failure and mode of failure were recorded: whether it was cohesive, adhesive or failure of the collagen substrate. Only adhesive failure was taken into account.

Compressive Modulus—Bioadhesive Elasticity:

Cylindrical samples (7.8 mm diameter, 3.4 mm height) were prepared in a silicon mold and analyzed 24 h after casting in order to measure the compressive modulus. The compressive elastic modulus (Ec) was measured using the above-described Instron machine. Cylindrical material samples were preconditioned by 3 cycles of loading/unloading following a ramped compressive displacement at a rate of 0.2 mm/min and a maximal strain of 35%. Five specimens were tested for each formulation. The compressive modulus (Ec) was calculated as the slope of the linear regression line for data between 15 to 25% of strain.

### X-Ray Diffraction (XRD):

XRD data were collected in symmetric Bragg-Brentano geometry with CuKα radiation on a Bruker D8 Discover (Germany) Θ:Θ X-ray diffractometer equipped with 1D LynxEye detector based on compound silicon strip technology, in order to measure the change in the gallery distance of MMT due to incorporation into the bioadhesive.

### Viscosity Measurements:

The initial viscosity of the polymeric (Gel-Al) bioadhesive at the moment of application and mixing on the tissue, prior to curing, is affected mainly by the viscosity of the aqueous Gel-Al solution. Viscosity measurements of polymer solutions were performed using a controlled stress rheometer (model DHR3, TA Instruments Ltd.) fitted with a cone-and-plate geometry (1° cone angle, 40 mm diameter), at a constant temperature of 25° C. or 37° C. (for fish or porcine gelatin, respectively) and a constant shear rate of 10 Hz, in order to investigate the effect of the hemostatic agents on the bioadhesive's initial viscosity.

### Gelation/Curing Time:

Crosslinking time, i.e. gelation and curing time, indicates the time required for the bioadhesive to reach the desired state when applied on a wound. Gelation time was determined as the time required for a magnetic bar to stop moving after mixing of the polymer solution with the crosslinker solution. Approximately 1 mL of bioadhesive, not loaded or loaded with a hemostatic agent, was poured into a 1.6 cm diameter plate under mixing at 300 rpm with a 1.4 cm magnetic bar at room temperature.

### Statistical Analysis:

All data were processed using the Excel software. Statistical comparison between more than two groups was performed using the ANOVA (with Tukey Kramer post hoc) method via the XLSTAT software. A value of p<0.05 was considered statistically significant. Error bars in the Figures indicate the standard error.

### Results and Discussion

In this study, the mechanical and physical properties of composite hydrogel bioadhesives based on fish gelatin and loaded with the hemostatic agents kaolin and MMT, were investigated. The effect of all components were studied, using three types of mechanical tests: XRD, viscosity and gelation time. A qualitative model describing the effect of all parameters on the cohesive and adhesive strengths is presented hereinbelow.

### Bioadhesive's Mechanical Properties:

The mechanical properties of the bioadhesive formulations were evaluated by three mechanical measurements selected based on the relevant standards.

### Effect of the Gelatin Concentration:

The burst pressure is defined as the maximum pressure that hydrogel adhesives can withstand before breaking with fluid leakage. When hydrogels are used as adhesives, hemostats or sealants, they are often subjected to significant pressure from underlying tissues or biological fluids.

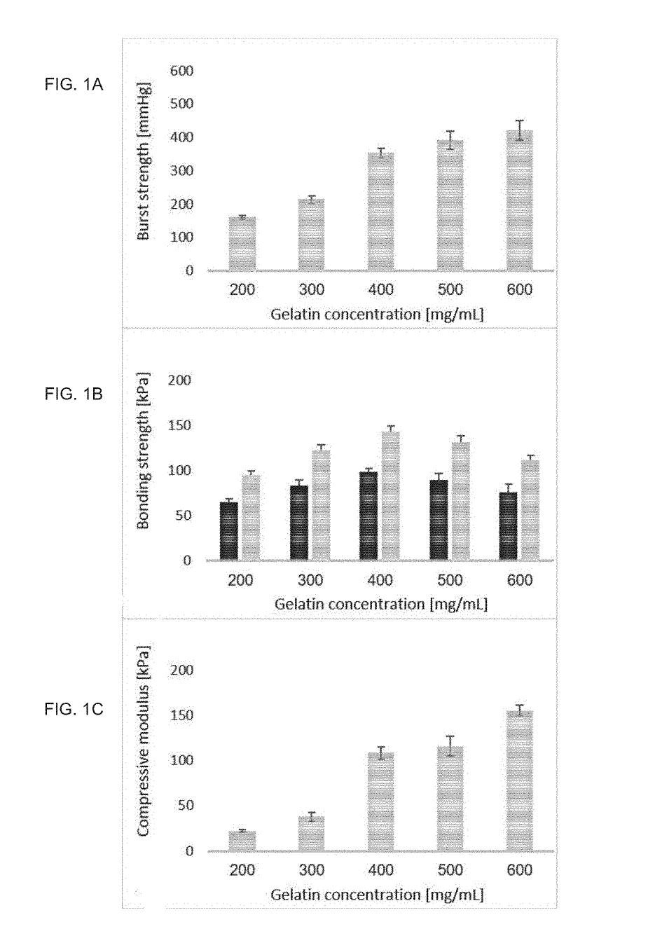

FIGS. 1A-C present comparative bar plots showing the effect of the gelatin concentration on: the burst strength (FIG. 1A), the bonding strength in lap shear also showing comparison between manual application of the bioadhesive (dark bars in FIG. 1B) and application by a double-syringe (light bars in FIG. 1B) and the elastic modulus in compression (FIG. 1C), wherein the EDC concentration was kept constant at 20 mg/mL.

As can be seen in FIG. 1A, the exemplary formulations exhibit the ability to resist pressures of at least 160 mmHg. Bioadhesives designed as arterial vascular sealants have to withstand a systolic blood pressure of about 200 mmHg, while the required burst pressure limit for hydrogels used as sealants for corneal incisions is 67 mmHg. The increase in the gelatin concentration from 200 to 400 mg/mL leads to a dramatic 120% increase in the burst strength. Further increases in the gelatin concentration only slightly improve the burst strength. On the other hand, a more moderate increase (51%) in the shear strength was achieved when the gelatin concentration was increased from 200 to 400 mg/mL (FIG. 1B). At higher gelatin concentrations the lap shear strength was reduced and resulted in equal strength for 200 and 600 mg/mL gelatin. FIG. 1B also shows a comparison between manual spreading and mixing of the bioadhesive, and application using a double-syringe. The lap shear strength obtained by application with the double-syringe exhibited approximately 45% higher strength than following manual mixing. This syringe was therefore selected for the entire study.

As can be seen in FIG. 1C a notable increase in the bioadhesive's elastic modulus in compression was achieved when the gelatin concentration was increased from 300 to 400 mg/mL. At gelatin concentrations lower than 300 mg/mL, the bioadhesive exhibited low modulus of elasticity, indicating an elastic hydrogel. Although elasticity evaluation is usually carried out by a tensile test, the compression test was selected in order to minimize the effect of defects in the molded specimens.

The results indicate that the crosslinking reaction reaches saturation at a gelatin concentration of approximately 400 mg/mL, i.e., the reactivity of EDC is fully occupied by the gelatin-alginate functional groups. Interestingly, at relatively low concentrations of gelatin (200 and 300 mg/mL), all the mechanical characteristics were relatively low despite the fact that the EDC:gelatin ratio was high, meaning a high crosslinker and functional groups (amine and carboxyl) ratio. It can therefore be assumed that the concentration of the functional groups in the aqueous solution has a greater effect on the crosslinking reaction than the ratio between the crosslinker and the functional groups.

The lap shear strength is influenced by several factors, which eventually determine the adhesion strength and the cohesion strength. Conversely, the elastic modulus of the bioadhesive correlates mainly with the bioadhesive's cohesive strength. The burst strength of the bioadhesive is also affected by the adhesive and cohesive strengths; however, the latter has a greater effect. The mild effect of the gelatin concentration on the lap shear strength with the maximum point can be explained by the integrated effect of the cohesive and adhesive forces. Up to 400 mg/mL, the cohesive strength increases significantly, as observed from the burst strength and the elastic modulus. The change in cohesive strength between the 200 and 400 mg/mL gelatin concentrations has a lower effect on the lap shear strength than the burst strength or the elastic modulus due to much better mechanical interlocking at the lower concentration as a result of lower viscosity. The decrease in the lap shear strength above 400 mg/mL gelatin is obtained as a result of a lower mechanical interlocking ability and the fact that at these concentrations, the cohesive strength increased rather moderately.

### Effect of the Alginate Concentration:

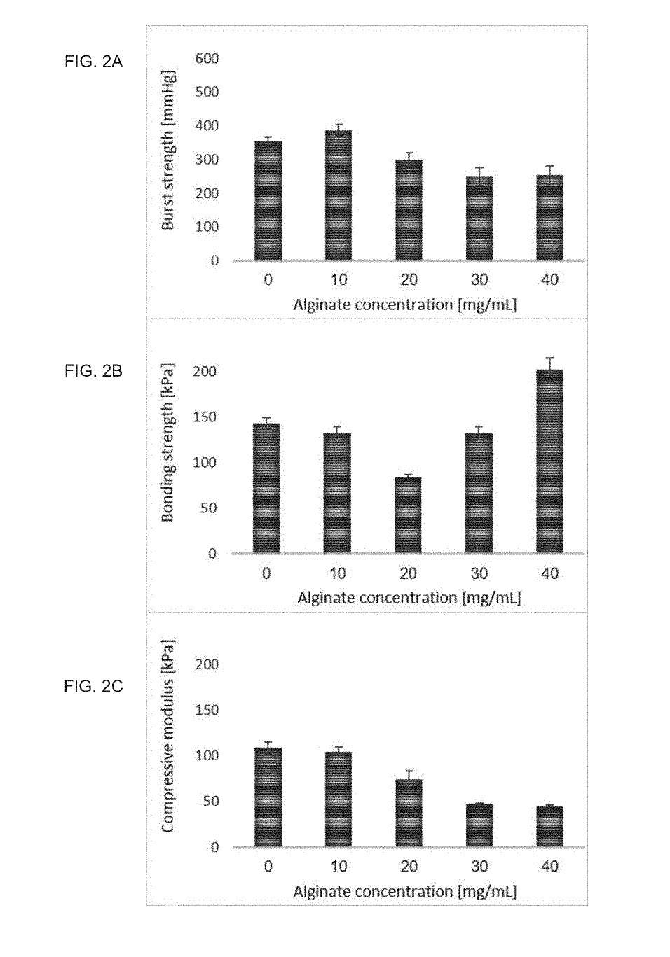

FIGS. 2A-C present comparative bar plots showing the effect of the alginate concentration on burst strength (FIG. 2A), the bonding strength in lap shear (FIG. 2B), and the elastic modulus in compression (FIG. 2C), of a bioadhesive based on 400 mg/mL gelatin and 20 mg/mL EDC.

The evaluation of the alginate effect on the bioadhesive's mechanical properties was conducted on a hydrogel solution with 400 mg/mL gelatin, which was found as a suitable concentration. As evident from FIG. 2A, the alginate concentration reduced the burst strength by approximately 30% when loaded at the maximal concentration of 40 mg/mL. Insignificant changes were observed at concentrations of 10 and 20 mg/mL. The crosslinked hydrogel exhibited lower modulus when the alginate concentration increased (FIG. 2C). It can thus be suggested that the bioadhesive's cohesion strength is reduced due to the incorporation of alginate.

It is known that polymer blend hydrogels with a good interaction between the two types of polymers result in higher degrees of chain entanglements. Theoretically, more entanglements result in a denser network and therefore induce higher cohesion strength. One reasonable explanation for the opposite results could be as follows: under a constant concentration of gelatin and EDC, increasing the alginate concentration reduced the crosslinking density since the relative portion of EDC in the adhesive matrix decreased and the carboxylic acid group portion increased. The crosslinking efficacy thus decreased with the increase in the alginate concentration.

On the other hand, although 20 mg/mL alginate reduced the lap shear strength by 40%, higher concentrations were found to improve the lap shear strength. The lap shear strength at the highest concentration of 40 mg/mL was found to be 40% higher than that of a gelatin solution without alginate. As mentioned above, the lap shear strength reflects both the adhesive and the cohesive strengths of bioadhesives. When assuming that change in the burst strength and the elastic modulus results in the decrease of the cohesive strength, the increase in lap shear strength at the higher alginate concentrations is evidence of a strong increase in the adhesive strength. This may be due to an improved interaction between the carboxylic group and the adherent. In general, improvements in adhesion strength found in in vitro evaluations of bioadhesives should be regarded with caution. Although these methods are standard bench tests, the adherent and the precise conditions do not closely simulate in vivo conditions. The composition of 400-10-20 mg/mL Gel-Al-EDC was selected for the following evaluation of the hemostatic agent's effect.

### Effect of the Hemostatic Agent Concentration:

The hemostatic agents MMT and kaolin were loaded into the bioadhesive at relatively high concentrations, which resulted in a considerable increase in the solution's viscosity. Kaolin was loaded to a maximal concentration of 50 mg/mL, whereas MMT was loaded up to 20 mg/mL.

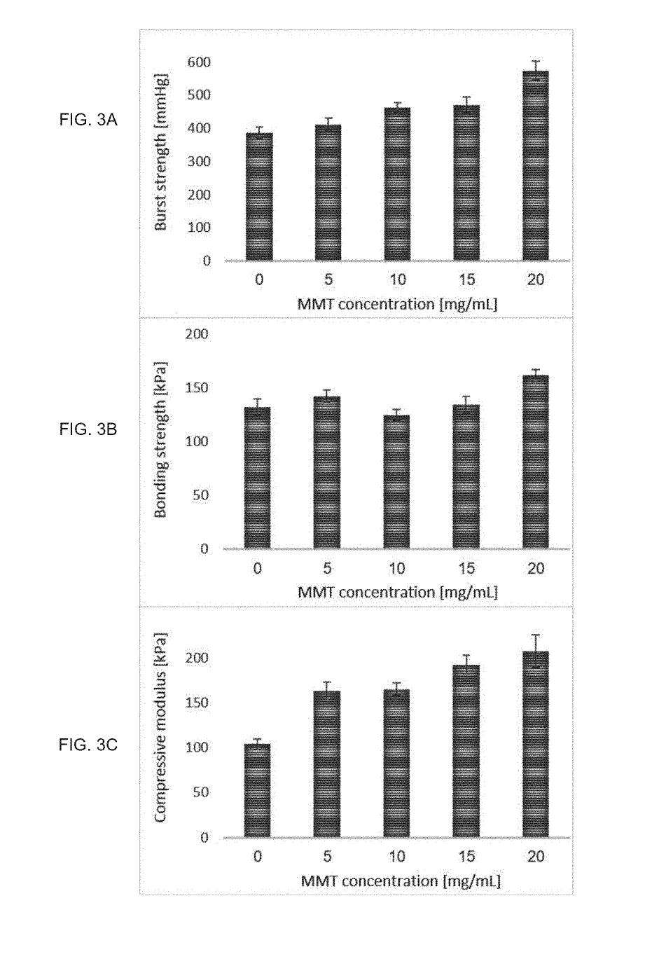

FIGS. 3A-C present comparative bar plots showing the effect of the MMT concentration on the burst strength (FIG. 3A), the bonding strength in lap shear (FIG. 3B); and the elastic modulus in compression (FIG. 3C), of a bioadhesive based on 400:10:20 Gel-Al-EDC.

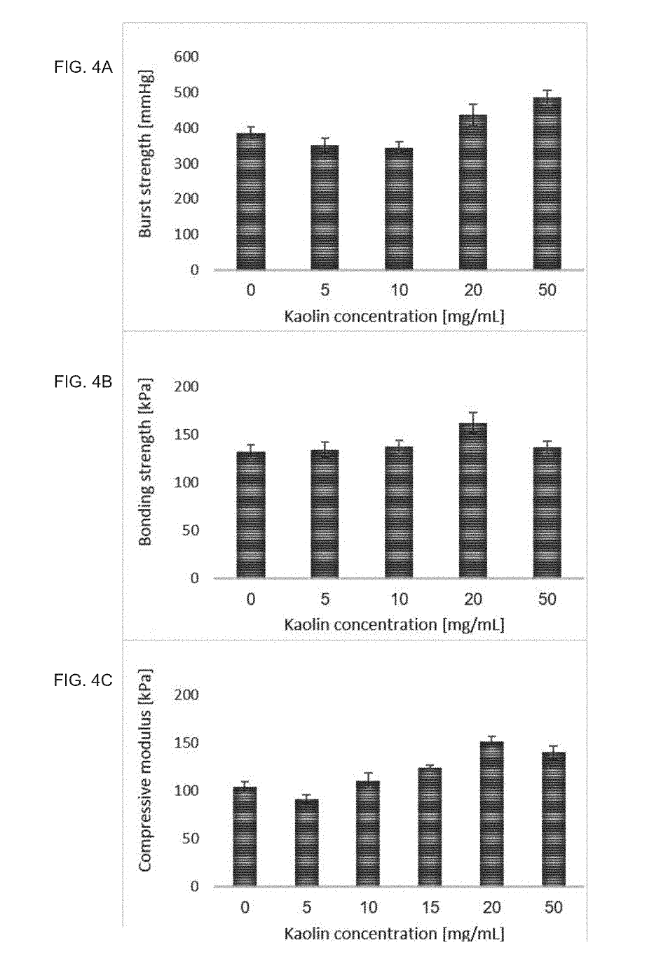

FIGS. 4A-C present comparative bar plots showing the effect of the kaolin concentration on the burst strength (FIG. 4A), the bonding strength in lap shear (FIG. 4B); and the elastic modulus in compression (FIG. 4C), of a bioadhesive based on 400:10:20 Gel-Al-EDC.

As can be seen in FIG. 3A, MMT was found to strongly affect the burst strength. An increase of approximately 53% was obtained when a MMT concentration of 20 mg/mL was used. As can be seen in FIG. 3C, a more prominent effect of the MMT was found on the elastic modulus. The elastic modulus increased up to 2-fold compared to the non-loaded formulation when using the highest concentration of 20 mg/mL. There was no significant enhancement of the lap shear strength when MMT was incorporated, and the lap shear strength was similar for all MMT concentrations.

As can be seen in FIGS. 4A-C, similar trends were observed when kaolin was loaded.

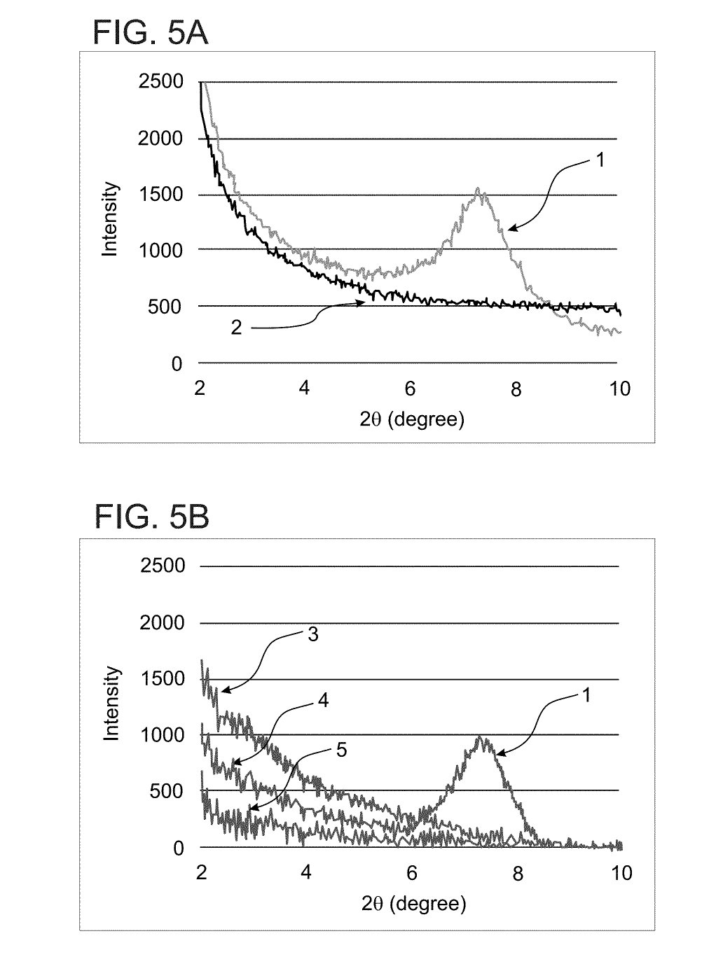

FIGS. 5A-B show the results of the XRD studies of MMT, wherein FIG. 5A shows the XRD patterns of pristine MMT (line No. 1) and unloaded bioadhesive (line No. 2), and FIG. 5B shows the normalized XRD patterns of a bioadhesive composite formulation, according to some embodiments of the present invention, loaded with 20 mg/ml MMT (line No. 3) 10 mg/ml MMT (line No. 4) and 5 mg/mL MMT (line No. 5).

A comparison of FIGS. 4A-C and FIGS. 5A-B shows that greater improvement resulted from the incorporation MMT than from kaolin. Kaolin had no effect on the lap shear strength, whereas a 25% enhancement in the burst strength was observed in the highest kaolin concentration. This moderate improvement in cohesion strength was thus expressed in a 50% increase in the elastic modulus compared to the unloaded bioadhesive.

The significant enhancement of the mechanical properties is attributed to a strong interaction between the gelatin and MMT, especially hydrogen interactions between carboxylate from gelatin and hydroxyl groups from MMT. In addition, a strong interaction exists between the positive amino acids in the gelatin (NH₃) and the negative sites in the MMT galleries. On the one hand, this interaction reduced the free amine groups, thus leading to a reduction of the carbodiimide chemical crosslinking efficiency. Furthermore, the stability of the entire polymeric matrix is enhanced by MMT which serves as a physical crosslinking agent.

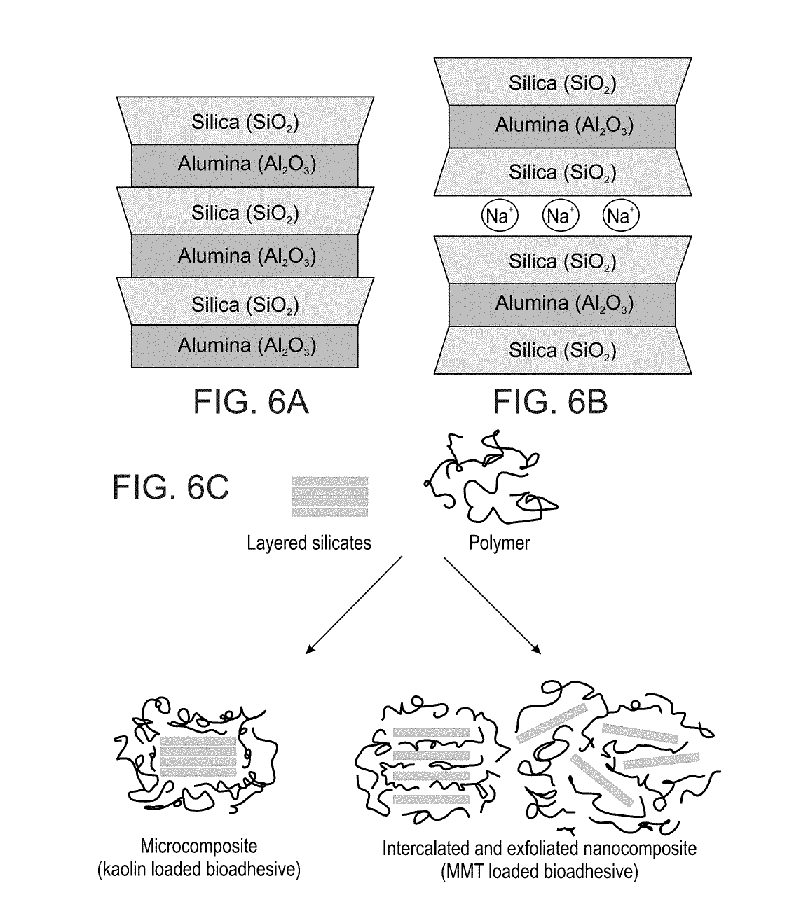

FIGS. 6A-C present schematic illustrations of the chemical structure of kaolin (FIG. 6A) and sodium montmorillonite (MMT; FIG. 6B), and the different types of polymer/layered silicate composites, wherein a microcomposite is suggested to characterize the kaolin silicate composites, and an intercalated nanocomposite and exfoliated nanocomposite is suggested to characterize the MMT silicate composites.

As can be seen in FIGS. 6A-C, although both hemostatic agents have similar crystalline structures, the composite structures that were created when the bioadhesive was loaded with the hemostatic agents differ greatly in their mechanical properties, especially in their cohesive strength. The interaction between kaolin and the polymeric matrix probably results from hydrogen bonds and electrostatic forces. However, kaolin is a non-expanding layered silicate and the interaction thus occurred only on the initial surface area. In contradistinction, MMT is an expanding layered silicate, which can greatly increase the silicate's surface area and can thus greatly increase the amount of interactions. These strong interactions lead to the stable nano-dispersion of MMT in the polymer matrix, yielding a nanocomposite structure such as intercalated and exfoliated composites.

In conclusion, the Gel-Al-EDC bioadhesive formulations present excellent mechanical properties. The gelatin concentration was found to be directly correlated to the bioadhesive's mechanical strength, whereas the integration of alginate could slightly decrease its strength, in particular the cohesive strength. Both hemostatic agents improved the mechanical strength of the bioadhesive to some extent; however, smaller quantities of MMT were needed compared to kaolin, indicating that the former is more effective. Furthermore, an extra enhancement of the bioadhesive's mechanical abilities may be revealed in animal studies as an improvement of the functionality in the hemorrhagic environment due the presence of hemostatic agents.

### Structural Features:

The XRD patterns of the pristine MMT and unloaded Gel-Al-EDC bioadhesive are presented in FIG. 5A. MMT showed a single peak at 2θ=7.3° corresponding to a d-spacing (001) of 1.2 nm in the silicate layer, according to the Bragg equation. FIG. 5B presents the normalized XRD patterns of pristine MMT and bioadhesive formulations loaded with various MMT loads and compared to the unloaded bioadhesive in order to elucidate changes due to nanocomposite formation. As can be seen in FIGS. 5A-B, the MMT loaded bioadhesive contains new broad peaks shifted to lower angles than the MMT (001) peak. This shifting may be due to the confinement of polymeric chains into the MMT gallery and indicate formation of a nanocomposite structure. The lack of the MMT (001) reflection may suggest a more exfoliated than intercalated structure.

Incorporation of layered silicate into a polymer matrix may result in a classic microcomposite with phase separation between the silicate and the polymer matrix. Such a structure is usually formed in a 1:1 layered silicate such as kaolin. On the other hand, nanocomposite structures can be formed due to the expansion of the layers. 2:1 layered silicates such as montmorillonite, which has weaker bonds between the layers, therefore, have the ability to expand under specific conditions. Partial expansion allows polymer chain intercalation between the silicate layers, i.e. an intercalated structure, illustrated in FIG. 6C. More extensive expansion is expressed as full delamination into singular layers, i.e. an exfoliated structure.

### Viscosity:

The bioadhesive's viscosity is a crucial characteristic, which determines the ease of use, and affects the mechanical properties through the mechanical interlocking mechanism. Rheological tests were performed in order to elucidate the effect of the bioadhesive's components on the initial viscosity, i.e. before the crosslinking reaction. The measurements were carried out on Gel-Al solutions at 25° C.

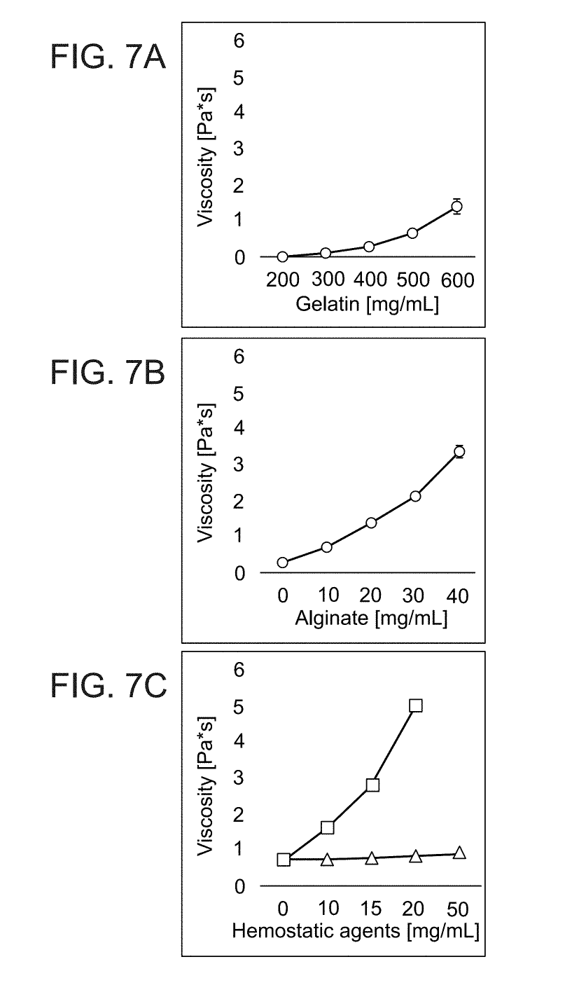

FIGS. 7A-C present the results of rheological tests conducted to assess the viscosity of exemplary bioadhesive formulations, according to some embodiments of the present invention, showing the effect of the concentration of gelatin (FIG. 7A), alginate based on 400 mg/mL gelatin (FIG. 7B) and the concentration of MMT (marked by squares in FIG. 7C) and kaolin (marked by triangles in FIG. 7C), in a bioadhesive formulation having 400:10 Gel-Al.

As can be seen in FIGS. 7A-C, the viscosity increased with the gelatin concentration—each additional 100 mg/mL of gelatin increased the viscosity by approximately 2.7 fold. The results presented in FIG. 7B demonstrate that alginate strongly increased the viscosity even when the concentration increment was an order of magnitude lower, i.e. 10 mg/mL.

As can be seen in FIG. 7C, the hemostatic agents exhibited different effects on the solution's viscosity. The hydrogel's viscosity increased up to 7 times when loaded with the highest amount of MMT, whereas loading of kaolin had no effect on the viscosity even though it was loaded at a 2.5 times higher concentration than MMT (50 mg/mL versus 20 mg/mL).

The increase in the Gel-Al solution's viscosity with the increase in MMT concentration is probably due to the expanded nature of the MMT layered silicate, which led to a massive increase in the polymeric matrix—layered silicate interfaces, which acted as a physical crosslinker, which stabilized the polymeric solution. Although kaolin and MMT create the same interaction with the matrix, kaolin is an unexpanded layered silicate and the interaction is insignificant for reinforcing the hydrogel.

When the viscosity of hydrogel is low, the mechanical interlocking ability is usually good, i.e. adhesion strength increases due to easier penetration of the hydrogel into the micropores in the tissue. High-viscosity bioadhesives usually exhibit higher chain entanglements and denser polymeric networks, which contribute to higher cohesion strength.

### Gelation Time:

Optimal gelation time depends on the clinical procedure and is usually in the range of 5-60 seconds.

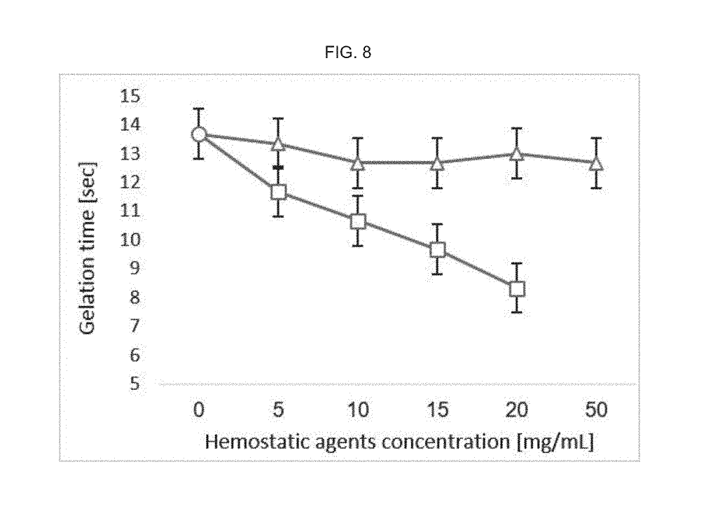

FIG. 8 present the gelation time of the 400:10:20 Gel-Al-EDC bioadhesive as affected by the concentration of MMT (marked by squares) and the concentration kaolin (marked by triangles), as used in an exemplary bioadhesive formulation based on 400:10:20 Gel-Al-EDC.

As can be seen in FIG. 8, all studied formulations exhibited gelation times in the desired range. The maximal MMT concentration accelerated gelation time by approximately 30%, whereas kaolin had no effect on the gelation time even when loaded at a high concentration of 50 mg/mL. These results are consistent with the mechanical strength results, shown in FIG. 4 and FIG. 5, and the viscosity results shown in FIG. 7, in which it was demonstrated that kaolin has almost no effect, probably due to less interaction with the polymeric matrix than MMT. Furthermore, the viscosity of the hydrogel and gelation time are interdependent. It should be noted that the gelation time of the studied bioadhesive can be further tuned by several means such as the polymeric or the crosslinker concentration, the pH, the double-syringe geometry and more.

### Effect of Gelatin Source on the Bioadhesive's Properties:

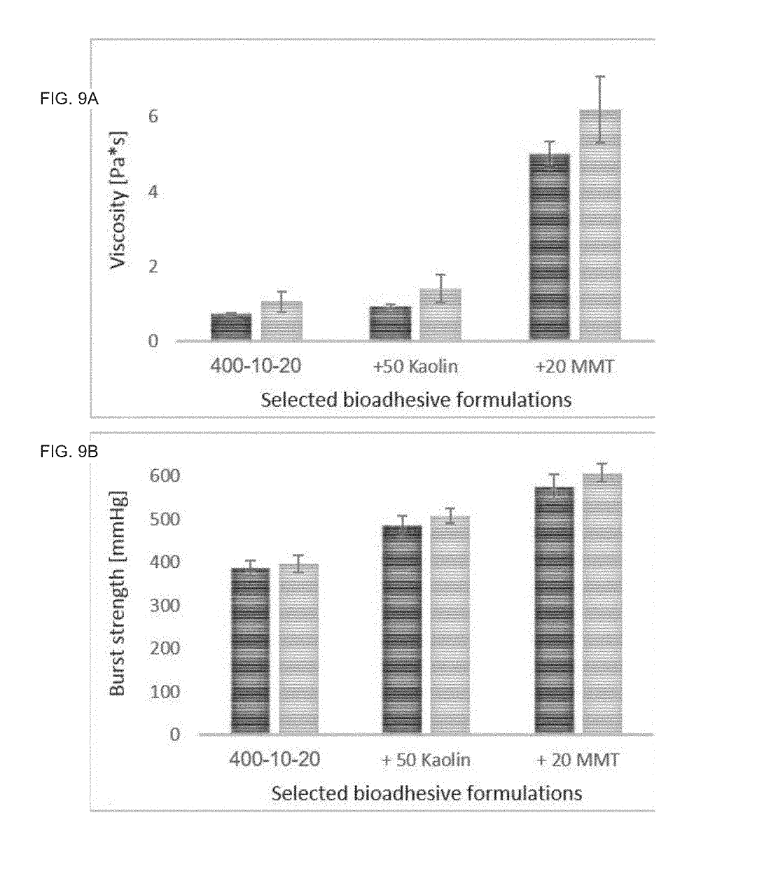

FIGS. 9A-B show the effect of coldwater fish gelatin (dark bars) versus porcine gelatin (light bars) on the initial viscosity (FIG. 9A) and the burst strength (FIG. 9B), measured for an exemplary bioadhesive formulation, according to some embodiments of the present invention, having Gel-Al-EDC 400-10-20 mg/mL.

As can be seen in FIGS. 9A-B, the initial viscosity of both unloaded and hemostatic agent-loaded bioadhesive formulations was not affected by the gelatin source. The burst strengths are also identical and practically not influenced by the gelatin source. Although porcine gelatin is much more common for biomedical application than coldwater fish gelatin, the results suggest that the carbodiimide crosslinking reaction is equally efficacious for gelatin from both sources. The added value of using coldwater fish gelatin as opposed to porcine gelatin is in eliminating the need for heating the polymeric hydrogel prior to the application. This can be useful for some medical applications.

### A Formulation—Strength Model:

A qualitative model that describes the effect of the bioadhesive formulation parameters on the cohesive and adhesive strength, through the relevant bioadhesive characteristics, is suggested, based on the above findings.

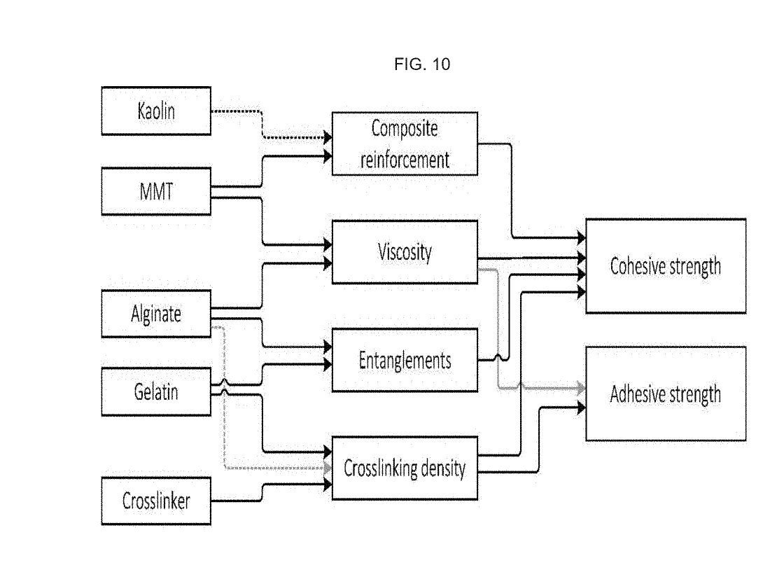

FIG. 10 presents a schematic illustration of a qualitative model describing the effects of the bioadhesive components on the cohesive and adhesive strength, wherein the dark/light arrows represent a case where an increase/decrease, respectively, in a certain parameter results in an increase in the following one, while the dashed lines represent a more moderate response.

According to this model, incorporation of MMT strongly affects the cohesive strength of the bioadhesive through its reinforcing effects and increased viscosity. A higher alginate content also affects the cohesive strength through enhanced viscosity and entanglements. Increasing the gelatin content of the bioadhesive enhances both cohesive strength and adhesive strength, through enhanced entanglements and crosslinking density, respectively. As expected, increasing the crosslinking agent content also contributes to higher adhesive strength, due to higher crosslinking density.

A bioadhesive's mechanical strength is a combination of the adhesive strength and the cohesive strength. When bioadhesives are used for gluing applications, they must be designed with high adhesion to the tissue. As described in our suggested model, use of a low-viscosity hydrogel is preferred in order to enhance the adhesion mechanism and enable penetration into the tissue's micropores. Furthermore, the crosslinking density needs to be high as possible. This can be achieved by choosing the appropriate polymers, crosslinking agent and reaction medium. An increase in crosslinking density can be reflected in a higher amount of intermolecular bonds between the polymeric chains or as an extra chemical interaction with the tissue.

The requirements for adhesion strength for surgical sealant applications are less substantial due to lower mechanical forces applied on the sealant; however, the internal strength, i.e. the cohesive strength of the polymeric hydrogel, is crucial for enabling the sealant plugs to resist the fluid flux without being ripped out. The cohesive strength of hydrogels can be tailored by adjusting polymeric matrix properties such as concentration and type of polymer, as well as molecular weight and modification. These can lead to changes in the viscosity and potential entanglements. Moreover, enhancing the cohesiveness of the matrix can be achieved by composite reinforcement which acts as physical crosslinking that stabilizes the polymeric matrix. Both hemostatic agents with layered silicate structures reinforce the polymeric matrix. The MMT bioadhesive, which is arranged at the nanocomposite level (as shown in the XRD, FIG. 5) enhances the mechanical properties significantly.

### CONCLUSIONS

Bioadhesive formulations, according to some embodiments of the present invention, based on gelatin and alginate crosslinked with EDC, were designed for potential adhesive and sealant applications. Incorporation of the hemostatic agents kaolin and MMT resulted in microcomposite and nanocomposite hydrogels, respectively, with enhanced mechanical and physical properties. The nanocomposite MMT-loaded hydrogel exhibited superior properties compared to the microcomposite kaolin-loaded hydrogel.

The gelatin content of the bioadhesive affects the burst strength, bonding strength and compression modulus. The two latter properties are also affected by the alginate content. The viscosity is affected mainly by the gelatin and MMT contents. The latter also strongly affects the gelation time.

Based on the suggested model, incorporation of MMT strongly affects the cohesive strength of the bioadhesive through its reinforcing effects and increased viscosity. A higher alginate content also affects the cohesive strength through enhanced viscosity and entanglements. Increasing the gelatin content of the bioadhesive enhances both cohesive strength and adhesive strength, through enhanced entanglements and crosslinking density, respectively. Coldwater fish gelatin was found to be advantageous compared to porcine gelatin, because it enables processing the bioadhesive at room temperature (instead of at an elevated temperature) with practically no change to the bioadhesive's properties.

Understanding the effects of a bioadhesive's components on its mechanical and physical properties, especially cohesive and adhesive strengths, together with structuring effects of these new composite adhesives, enables adjusting the relevant parameters for various applications.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A kit for forming a bioadhesive, comprising a first container containing a first formulation and a second container containing a second formulation, said first formulation comprises gelatin and alginate and said second formulation comprises a coupling agent for coupling said gelatin and/or for coupling said alginate and/or for coupling said gelatin to said alginate, wherein at least one of said first formulation and said second formulation comprises montmorillonite.

2. The kit of claim 1, wherein a concentration of said gelatin in a bioadhesive obtained by combining said first formulation and said second formulation at volume ratio of 1:9 to 25:1, ranges from 50 mg/ml to 500 mg/ml.

3. The kit of claim 1, wherein a concentration of said alginate in a bioadhesive obtained by combining said first formulation and said second formulation at volume ratio of 1:9 to 25:1, ranges from 5 mg/ml to 100 mg/ml.

4. The kit of claim 1, wherein a concentration of said montmorillonite in a bioadhesive obtained by combining said first formulation and said second formulation at volume ratio of 1:9 to 25:1, ranges from 1 mg/ml to 50 mg/ml.

5. The kit of claim 1, wherein a concentration of said coupling agent in a bioadhesive obtained by combining said first formulation and said second formulation at volume ratio of 1:9 to 25:1, ranges from 1 mg/ml to 40 mg/ml.

6. The kit of claim 1, wherein, in a bioadhesive obtained by combining said first formulation and said second formulation at volume ratio of 1:9 to 25:1, a concentration of said gelatin ranges from 200 mg/ml to 400 mg/ml, a concentration of said alginate ranges from 20 mg/ml to 40 mg/ml, a concentration of said montmorillonite ranges from 5 mg/ml to 30 mg/ml and a concentration of said coupling agent ranges from 10 mg/ml to 30 mg/ml.

7-8. (canceled)

9. The kit of claim 1, wherein a concentration of said gelatin in a bioadhesive obtained by combining said first formulation and said second formulation at volume ratio of 1:9 to 25:1, is less than 500 mg/ml, and said bioadhesive is characterized by a room temperature viscosity that ranges from 1 Pa-sec to 50 Pa-sec upon said combining and up to 30 minutes from said combining.

10. The kit of claim 1, wherein said first formulation and/or said second formulation further comprises a crosslinking promoting agent.

11. The kit of claim 1, wherein said first formulation and/or said second formulation further comprises a bioactive agent.

12. (canceled)

13. The kit of claim 1, being for forming a bioadhesive matrix upon curing, wherein a curing time for forming said matrix ranges from 5 seconds to 30 minutes.

14. The kit of claim 13, wherein said matrix is characterized by a burst strength expressed in a maximal pressure required to rupture a layer of said matrix having a thickness of about 1 mm and afforded by applying about 0.5 ml of said bioadhesive over and around a hole of about 3.0 mm uniform diameter punched in a collagen sheet, according to Standard Test Method for Burst Strength of Surgical Sealants ASTM F2392-04, said maximal pressure ranges from 350 mmHg to 650 mmHg.

15. The kit of claim 1, being an applicator device for dispensing said first formulation from said first container and said second formulation from said second container to thereby form the bioadhesive.

16. The kit of claim 15, wherein said applicator comprises:
said first container in a form of a first syringe having a first barrel defining a first chamber for retaining said first formulation, and a first plunger having one end received in said chamber for extruding said first formulation from said first chamber;
said second container in a form of a second syringe having a second barrel defining a second chamber for retaining said second formulation, and a second plunger having one end received in said second chamber extruding said second formulation from said second chamber;
a nozzle having a distal end, a proximal end, and a lumen extending through said nozzle and means for connecting said proximal end of said nozzle to said first chamber and said second chamber such that said first formulation and said second formulation come in contact in said lumen,
whereby forming the bioadhesive that may be ejected through said nozzle upon driving said first plunger and said second plunger.

17. The kit of claim 1, identified for use in adhering a biological object.

18. The kit of claim 1, identified for use in sealing a rupture in a biological object.

19. The kit of claim 1, identified for use in bonding at least two objects to one another, at least one of the objects being a biological object.

20-23. (canceled)

24. A bioadhesive matrix, formed by contacting a first formulation that comprises gelatin and alginate, and a second formulation that comprises a coupling agent for coupling said gelatin and/or for coupling said alginate and/or for coupling said gelatin to said alginate, wherein at least one of said first formulation and said second formulation comprises montmorillonite.

25. The bioadhesive matrix of claim 24, further comprising a bioactive agent sequestered therein, the bioadhesive matrix is a drug-eluting bioadhesive matrix.

26. A bioadhesive comprising:
a) gelatin;
b) alginate;
c) montmorillonite;
d) a coupling agent for coupling said gelatin and/or for coupling said alginate and/or for coupling said gelatin to said alginate; and
e) water.
